(19) United States
(12) Patent Application Publication
(10) Pub. No.: US 2011/0224229 A1
(43) Pub. Date: Sep. 15, 2011

(54) Novel Crystalline Form

(75) Inventor: Martin Lindsjö, Lund (SE)

(73) Assignee: AstraZeneca AB, Soedertalje (SE)

(21) Appl. No.: 13/043,697

(22) Filed: Mar. 9, 2011

Related U.S. Application Data
(60) Provisional application No. 61/312,424, filed on Mar. 10, 2010.

(51) Int. Cl. A61K 31/497; C07D 403/04; A61P 11/00; A61P 17/06; A61P 29/00; A61P 9/10; A61P 35/00; A61P 1/04

(52) U.S. Cl. 514/255.05; 544/405

(57) ABSTRACT

Crystalline Forms of 6-[2-(4-cyano-phenyl)-2H-pyrazol-3-yl]-5-methyl-3-oxo-4-(3-trifluoromethyl-phenyl)-3,4-dihydro-pyrazine-2-carboxylic acid ethylamide are disclosed together with processes for preparing the Forms, pharmaceutical compositions comprising the Forms, and the use of the Forms in therapy.

### CROSS REFERENCE TO RELATED APPLICATIONS

The present application claims benefit under 35 U.S.C. §119(e) of U.S. Provisional Application Ser. No. 61/312,424 filed on Mar. 10, 2010.

### FIELD OF THE INVENTION

The present invention relates to a novel crystalline forms of 6-[2-(4-cyano-phenyl)-2H-pyrazol-3-yl]-5-methyl-3-oxo-4-(3-trifluoromethyl-phenyl)-3,4-dihydro-pyrazine-2-carboxylic acid ethylamide, processes for preparing the forms, pharmaceutical compositions containing the forms and the use of the forms in therapy.

### BACKGROUND TO THE INVENTION

Elastases are possibly the most destructive enzymes in the body, having the ability to degrade virtually all connective tissue components. The uncontrolled proteolytic degradation by elastases has been implicated in a number of pathological conditions.

The most important endogenous inhibitor of human neutrophil elastase (NE) is α₁-antitrypsin. The imbalance between human NE and antiprotease is believed to give rise to an excess of human NE resulting in uncontrolled tissue destruction. The protease/antiprotease balance may be upset by a decreased availability of α₁-antitrypsin either through inactivation by oxidants such as cigarette smoke, or as a result of genetic inability to produce sufficient serum levels (α₁-antitrypsin deficiency). Human NE has been implicated in the promotion or exacerbation of a number of diseases as described in for example, WO2007/129963. Examples of diseases, which may benefit from treatment with an inhibitor of neutrophil elastase include adult respiratory distress syndrome (ARDS), cystic fibrosis, pulmonary emphysema, bronchitis including chronic bronchitis, bronchiectasis, chronic obstructive pulmonary disease (COPD), pulmonary hypertension, asthma including refractive asthma, rhinitis, psoriasis, ischemia-reperfusion injury, rheumatoid arthritis, osteoarthritis, systemic inflammatory response syndrome (SIRS), chronic wound, cancer, atherosclerosis, peptic ulcers, Crohn's disease, ulcerative colitis or gastric mucosal injury.

Alpha-1-antitrypsin deficiency (AATD) is a genetic disorder which results in low serum levels of alpha-1 antitrypsin. Patients with AATD are prone to develop a number of diseases including lung disease such as emphysema and COPD, liver disease such as cirrhosis and the skin disease panniculitis. Patients with AATD are particularly prone to develop lung diseases such as COPD, emphysema and bronchitis. These conditions are likely to be accelerated when patients with AATD are exposed to environmental factors such as cigarette smoking, and dust exposure. A number of treatments for AATD have been approved including Prolastin®, Araslast° and Zemaira°. These treatments are all proteins which are administered to patients intravenously to increase the levels of alpha-1-antitrypsin, or derivatives thereof, in the serum. However, there remains a need to identify alternative treatments for patients with AATD.

WO2007/129963, which is incorporated herein by reference in its entirety, teaches a class of neutrophil elastase inhibitors that are useful in therapy. WO2007/129963 further discloses as Example 3, a specific neutrophil elastase inhibitor compound identified therein as 6-[2-(4-Cyano-phenyl)-2H-pyrazol-3-yl]-5-methyl-3-oxo-4-(3-trifluoromethyl-phenyl)-3,4-dihydro-pyrazine-2-carboxylic acid ethylamide. This compound if hereinafter “Compound (I)” and has the structure:

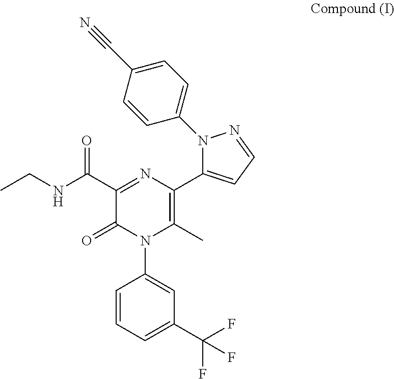

Compound (I) is a potent neutrophil elastase inhibitor and as such is expected to be useful in therapy. We have found that Compound (I) can be prepared as a novel crystalline form with advantageous properties.

### BRIEF DESCRIPTION OF THE DRAWINGS

FIG. 1 is an X-ray powder diffraction diagram of Compound (I) Form A measured under controlled conditions of 5% relative humidity and 25° C. (measured using CuKα1 radiation (1.5406 Å, 45 kV, 40 mA). The x-axis shows the 2-theta value and the y-axis the intensity.

FIG. 2 is a differential scanning calorimetry (DSC) trace for Compound (I) Form A. The x-axis shows temperature (° C.) and the y-axis heat flow (watts/g).

FIG. 3 is an X-ray powder diffraction diagram of Compound (I) Form B (measured using nickel-filtered CuK-radiation (1.5418 Å, 45 kV, 40 mA) under ambient conditions). The x-axis shows the 2-theta value and the y-axis the intensity.

FIG. 4 is a differential scanning calorimetry (DSC) trace for Compound (I) Form B. The x-axis shows temperature (° C.) and the y-axis heat flow (watts/g).

FIG. 5 is an X-ray powder diffraction diagram of Compound (I) Form A (bottom trace) and Compound (I) Form B (top trace) (measured on the same instrument using nickel-filtered CuK-radiation (1.5418 Å, 45 kV, 40 mA) under ambient conditions). The x-axis shows the 2-theta value and the y-axis the intensity.

### DETAILED DESCRIPTION OF THE INVENTION

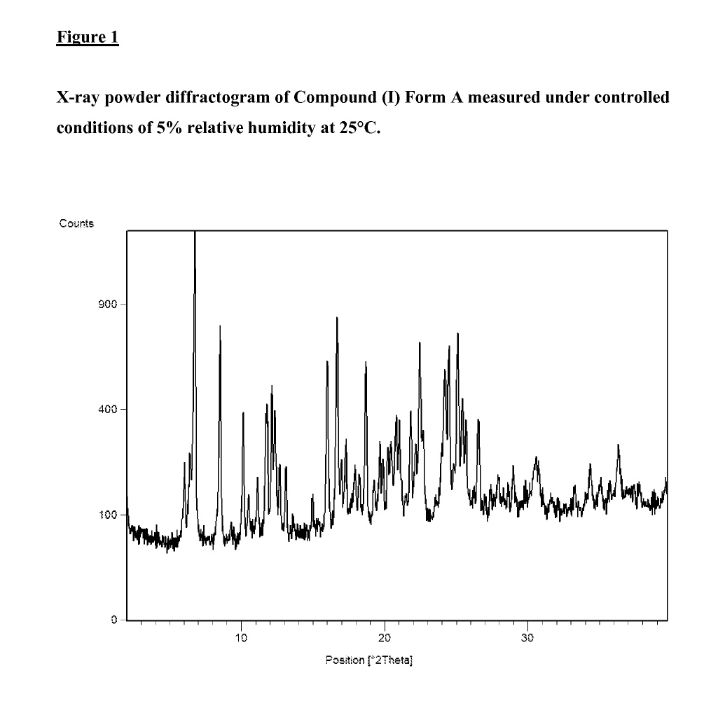

We have found that Compound (I) can be prepared in one crystalline form by crystallising the compound from ethanol, and certain other solvents described hereafter. This form of Compound (I), hereafter “Compound (I) Form A” is crystalline and provides an X-ray powder diffraction pattern substantially as shown in FIG. 1 when measured under controlled conditions of 5% relative humidity at 25° C., as described in the Examples. The most prominent peaks (2θ value) of Compound (I) Form A are shown in Table 1.

TABLE 1
Angle
2-Theta (2θ)°

6.0
6.7
8.5
10.1
11.1
12.1
12.3
13.1
16.0
16.7
17.3
18.7
22.4
24.5
25.1

Unless stated otherwise, the X-ray powder diffraction patterns described herein for Form A were measured using a PANalytical X'Pert PRO MPD theta-theta system, equipped with a focusing beam Johansson monochromator and an X'Celerator detector, using CuKα1 radiation (1.5406 Å, 45 kV, 40 mA), under controlled temperature and humidity conditions of 5% relative humidity and 25° C., as described in the Examples section.

According to one aspect of the invention there is provided Compound (I) Form A.

Accordingly Compound (I) Form A is characterised in that said Form A has an X-ray powder diffraction pattern with at least one specific peak at 2θ about =10.1 or 18.7°.

In one embodiment Compound (I) Form A is characterised in that said Form A has an X-ray powder diffraction pattern with at least one specific peak at 2θ about =10.1, 18.7, 22.4 or 25.1°.

In another embodiment Compound (I) Form A is characterised in that said Form A has an X-ray powder diffraction pattern with specific peaks at 2θ about =10.1 and 18.7°

In another embodiment Compound (I) Form A is characterised in that said Form A has an X-ray powder diffraction pattern with specific peaks at 2θ about =6.7, 10.1 and 18.7°.

In another embodiment Compound (I) Form A is characterised in that said Form A has an X-ray powder diffraction pattern with specific peaks at 2θ about =10.1, 18.7, 22.4 and 25.1°.

In another embodiment Compound (I) Form A is characterised in that said Form A has an X-ray powder diffraction pattern with specific peaks at 2θ about =6.7, 8.5, 10.1 and 18.7°.

In another embodiment Compound (I) Form A is characterised in that said Form A has an X-ray powder diffraction pattern with specific peaks at 2θ about =6.7, 8.5, 16.0, 16.7, 18.7, 22.4 and 25.1°.

In another embodiment Compound (I) Form A is characterised in that said Form A has an X-ray powder diffraction pattern with specific peaks at 2θ about =6.7, 8.5, 12.1, 12.3, 13.1, 16.0, 16.7, 18.7, 22.4, 24.5 and 25.1°.

In another embodiment Compound (I) Form A is characterised in that said Form A has an X-ray powder diffraction pattern with specific peaks at 2θ about =the values shown in Table 1.

Compound (I) Form A may also be characterised in that said Form A has an X-ray powder diffraction pattern substantially as shown in FIG. 1.

Compound (I) Form A is crystalline. Suitably, Compound (I) Form A is substantially free from other crystalline and non-crystalline Forms of Compound (I).

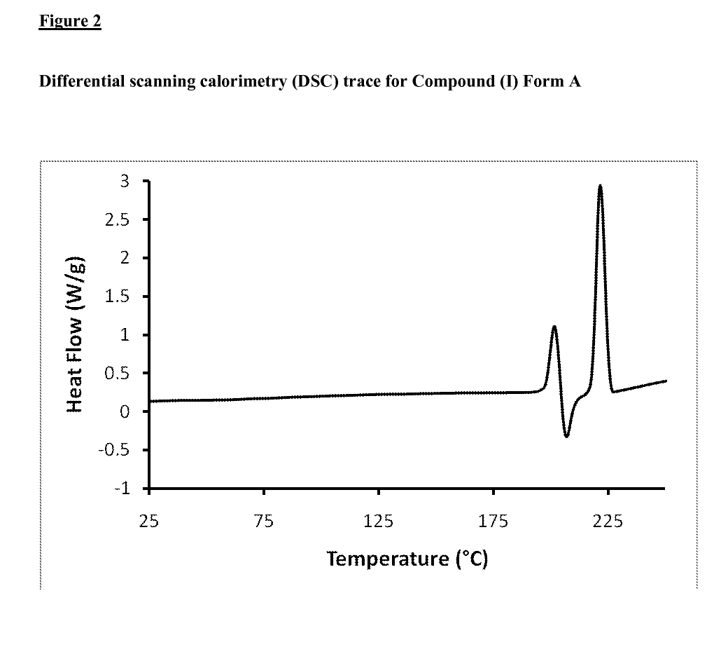

When heated in a Differential Scanning calorimeter (DSC) (conditions as described in the Examples section) Compound (I) Form A exhibits a melting endotherm with an onset temperature at about 198° C., as illustrated in FIG. 2.

We have found that Compound (I) Form A can form solvates (including hydrates), accordingly references herein to Compound (I) Form A are intended to include all solvated and hydrated forms of Compound (I) Form A.

Humidity sorption measurements using gravimetrical vapour sorption (GVS as described in the Examples Section) showed that dried Compound (I) Form A has a water uptake of about 1.6% by weight following exposure to 80% relative humidity (RH). As such, Compound (I) Form A is slightly hygroscopic.

When slurried in water, ethanol or acetonitrile we have found that Compound (I) Form A forms variable, non-stoichiometric solvates with acetonitrile or ethanol and hydrates with water. XRPD studies on the solvates and hydrates of Compound (I) Form A show that the positions of the peaks of the XRPD pattern vary slightly as the level of solvent or water in the crystal changes. Without wishing to be bound by theory, it is thought that the observed shifts in the XRPD pattern of Compound (I) Form A are produced by the crystal structure expanding to allow for uptake/release of solvent molecules without a major structural rearrangement of the crystal. Accordingly, the solvated/hydrated forms of Compound (I) Form A are thought to be channel hydrates/solvates resulting from the specific crystalline structure of Form A. This may also explain why this Compound (I) Form A is slightly hygroscopic.

According to a further aspect of the invention there is provided a solvate of Compound (I) Form A selected from a methanol, ethanol and acetonitrile solvate of Compound (I) Form A.

According to a further aspect of the invention there is provided a hydrate of Compound (I) Form A, which hydrate contains up to about 2% by weight (suitably up to about 1.8% by weight) water.

The tendency for Compound (I) Form A to form variable hydrates/solvates may be problematic in some applications. For example, Form A may be prone to retaining solvents present in the manufacturing process within the crystal structure which could be difficult to remove by drying thereby resulting in undesirable impurities in the product.

We have found another crystalline form of Compound (I), Compound (I) Form B. Compound (I) Form B is only formed under certain conditions; by heating Form A to high temperature; or by crystallisation from certain specific solvents. Compound (I) Form B is highly crystalline, thermodynamically stable and is substantially non-hygroscopic.

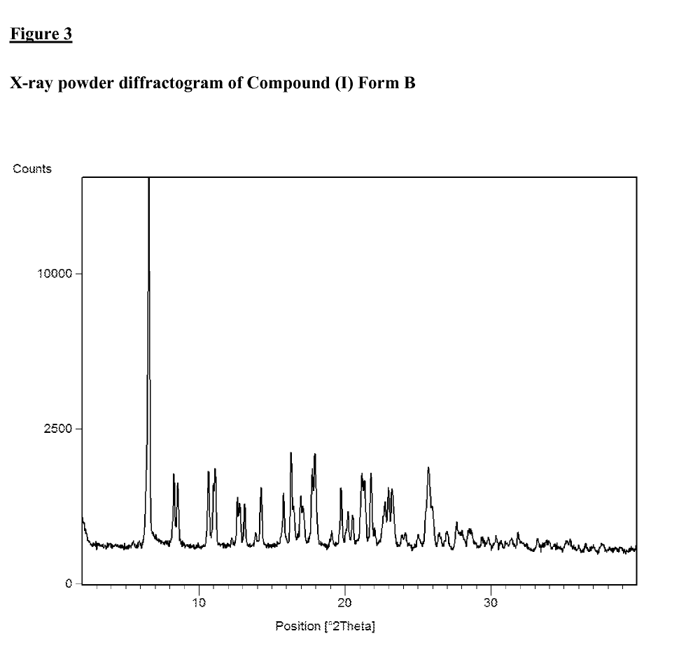

Compound (I) Form B is crystalline and provides an X-ray powder diffraction pattern substantially as shown in FIG. 3. The most prominent peaks (2θ value) of Compound (I) Form B are shown in Table 2.

TABLE 2
Angle
2-Theta(2θ)°

6.6
8.3
10.6
11.1
12.7
14.3
15.8
16.3
17.0
17.8
18.0
19.8
21.8
23.0
23.2
25.7

Unless stated otherwise, the X-ray powder diffraction patterns described herein for Form B were measured using a PANalytical X'Pert PRO MPD theta-theta system using nickel-filtered CuK-radiation (1.5418 Å, 45 kV, 40 mA) and an X'Celerator detector as described in more detail in the Examples section.

According to one aspect of the invention there is provided Compound (I) Form B.

According to one embodiment of the invention there is provided Compound (I) Form B, characterised in that said Form B has an X-ray powder diffraction pattern with at least one specific peak at 2θ about =14.3 or 23.2°.

According to one embodiment of the invention there is provided Compound (I) Form B, characterised in that said Form B has an X-ray powder diffraction pattern with at least one specific peak at 2θ about =14.3, 17.8 or 23.2°.

According to one embodiment of the invention there is provided Compound (I) Form B, characterised in that said Form B has an X-ray powder diffraction pattern with specific peaks at 2θ about =14.3 and 23.2°.

According to one embodiment of the invention there is provided Compound (I) Form B, characterised in that said Form B has an X-ray powder diffraction pattern with specific peaks at 2θ about =14.3, 17.8 and 23.2°.

According to one embodiment of the invention there is provided Compound (I) Form B, characterised in that said Form B has an X-ray powder diffraction pattern with specific peaks at 2θ about =6.6, 14.3 and 23.2°.

According to one embodiment of the invention there is provided Compound (I) Form B, characterised in that said Form B has an X-ray powder diffraction pattern with specific peaks at 2θ about =6.6, 14.3, 17.8 and 23.2°.

According to one embodiment of the invention there is provided Compound (I) Form B, characterised in that said Form B has an X-ray powder diffraction pattern with specific peaks at 2θ about =6.6, 8.3, 14.3, 16.3, 17.8 and 23.2°.

According to another embodiment of the invention there is provided Compound (I) Form B, characterised in that said Form B has an X-ray powder diffraction pattern with specific peaks at 2θ about =6.6, 8.3, 10.6, 11.1, 14.3, 16.3, 17.8, 19.8, 23.2 and 25.7°.

According to another embodiment of the invention there is provided Compound (I) Form B, characterised in that said Form B has an X-ray powder diffraction pattern with specific peaks at 2θ about =6.6, 8.3, 10.6, 11.1, 12.7, 14.3, 15.8, 16.3, 17.0, 17.8, 18.0, 21.8, 23.0, 23.2 and 25.7°.

According to one embodiment of the invention there is provided Compound (I) Form B, characterised in that said Form B has an X-ray powder diffraction pattern with specific peaks at 2θ about =to the values shown in Table 2.

According to one embodiment of the invention there is provided Compound (I) Form B, characterised in that said Form B has an X-ray powder diffraction pattern substantially as shown in FIG. 3.

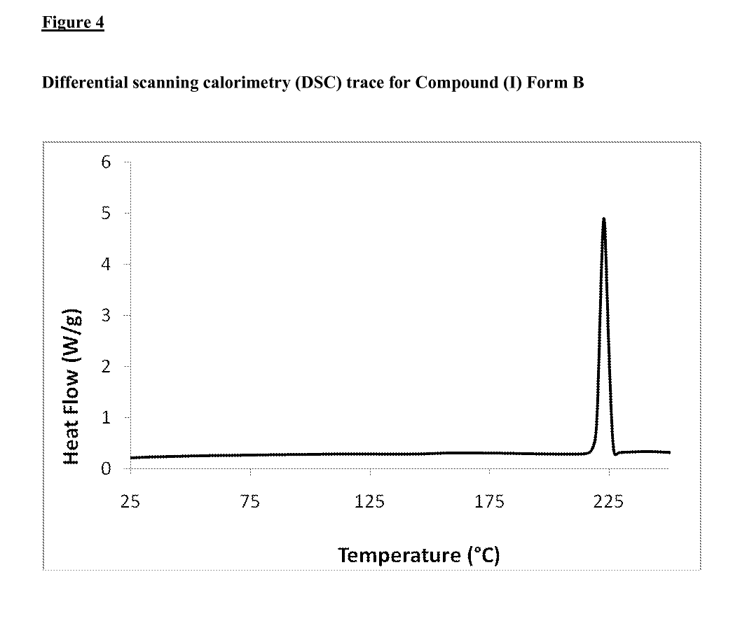

When heated in a Differential Scanning calorimeter (DSC) (conditions as described in the Examples section) Compound (I) Form B exhibits a melting/degradation endotherm with an onset temperature at about 220° C., as illustrated in FIG. 4

Humidity sorption measurements using gravimetrical vapour sorption (GVS as described in the Examples Section) showed Compound (I) Form B to have a water uptake of about 0.08% by weight following exposure to 80% relative humidity (RH). As such, Compound (I) Form A is non-hygroscopic.

Suitably, Compound (I) Form B is substantially free from other crystalline and non-crystalline Forms of Compound (I). For example, Compound (I) Form B is substantially free of Compound (I) Form A.

When slurried in water for 4 weeks Compound (I) Form B did not form hydrates.

The high crystallinity, stability and non-hygroscopicity of Compound (I) Form B make this particular Form suitable for use in the preparation of pharmaceutical compositions for use in therapy.

Suitably a crystalline Form of Compound (I) such as Form A or Form B is substantially free from other Forms of Compound (I). For example, a described crystalline Form of Compound (I) suitably includes less than 20%, 15%, 10%, 5%, 3% or particularly, less than 1% by weight of other crystalline and non-crystalline forms of Compound (I). For example the Compound (I) Form B is suitably includes less than about 10%, 5%, 3% or particularly, less than 1% by weight of Compound (I) Form A. In another embodiment, Compound (I) Form A is suitably includes less than about 10%, 5%, 3% or particularly, less than 1% by weight of Compound (I) Form B.

When herein reference is made to a Form of Compound (I) being crystalline, such as Compound (I) Form A or Form B, suitably the degree of crystallinity as determined by X-ray powder diffraction data, is for example greater than about 60%, such as greater than about 80%, particularly greater than about 90%, more particularly greater than about 95%. In embodiments of the invention, the degree of crystallinity as determined by X-ray powder diffraction data is greater than about 98%, wherein the % crystallinity refers to the % by weight of the total sample mass which is crystalline.

In the preceding paragraphs defining the X-ray powder diffraction peaks for the crystalline Forms of Compound (I), the term “about =” is used in the expression “ . . . at 2θ about = . . . ” to indicate that the precise position of peaks (i.e. the recited 2-theta angle values) should not be construed as being absolute values because, as will be appreciated by those skilled in the art, the precise position of the peaks may vary slightly between one measurement apparatus and another, from one sample to another, or as a result of slight variations in measurement conditions utilised. It is also stated in the preceding paragraphs that the Compound (I) Forms A and B provide X-ray powder diffraction patterns ‘substantially’ the same as the X-ray powder diffraction patterns shown in FIGS. 1 and 3 respectively, and has substantially the most prominent peaks (2-theta angle values) shown in Tables 1 and 2. It is to be understood that the use of the term ‘substantially’ in this context is also intended to indicate that the 2-theta angle values of the X-ray powder diffraction patterns may vary slightly from one apparatus to another, from one sample to another, or as a result of slight variations in measurement conditions utilised, so the peak positions shown in the Figures or quoted in the Tables are again not to be construed as absolute values.

The person skilled in the art of X-ray powder diffraction will realize that the relative intensity of peaks can be affected by, for example, grains above approximately 30 micrometer in size and non-unitary aspect ratios which may affect analysis of samples. Furthermore, it should be understood that intensities may fluctuate depending on experimental conditions and sample preparation such as preferred orientation of the particles in the sample. The use of automatic or fixed divergence slits will also influence the relative intensity calculations. A person skilled in the art can handle such effects when comparing diffraction patterns.

The person skilled in the art of X-ray powder diffraction will also realize that due to difference in sample heights and errors in the calibration of the detector position, a small shift in the 2θ positions could occur. Generally, a difference of ±0.2° from the given value are to be considered correct. These error tolerances equate to typically a sample height difference of 1 mm.

The person skilled in the art will also appreciate that slight variations in the melting point measured by DSC may occur as a result of variations in sample purity, sample preparation and the measurement conditions (e.g. heating rate). It will be appreciated that alternative readings of melting point may be given by other types of equipment or by using conditions different to those described hereinafter. Hence the melting point and endotherm figures quoted herein are not to be taken as absolute values and such measurement errors are to be taken into account when interpreting DSC data. Typically, melting points may vary by ±5° C. or less.

The crystalline Forms of Compound (I) may also be characterised and/or distinguished from other physical forms using other suitable analytical techniques, for example NIR spectroscopy or solid-state nuclear magnetic resonance spectroscopy.

The chemical structure of Compound (I) can be confirmed by routine methods for example proton nuclear magnetic resonance (NMR) analysis.

### Preparation of Compound (I) Form A

Compound (I) Form A may be prepared by crystallising Compound (I) from ethanol. It may also be possible to prepare Compound (I) Form A by crystallisation from methanol, or from a mixture of methanol, acetonitrile and water.

Examples of the preparation of Compound (I) Form A are illustrated in the Examples.

### Preparation of Compound (I) Form B

Compound (I) Form B may be prepared directly from Compound (I) Form A by heating Form A. The Form A is heated until it melts, Compound (I) Form B then crystallizes from the melt.

According to a further aspect of the invention there is provided a process for the preparation of Compound (I) Form B comprising heating Compound (I) Form A until the Form A melts; and crystallising Compound (I) Form B. Crystallisation of Compound (I) Form B from the melt is typically observed at a temperature in the range of from 200-220° C.

Compound (I) Form B may also be prepared by crystallisation from certain solvents.

Accordingly as a further feature of the present invention there is provided a process for the preparation of Compound (I) Form B comprising crystallising Compound (I) Form B from a solution of Compound (I) in methyl iso-butyl ketone (or 4-methylpentan-2-one, hereafter MIBK).

The crystallisation Compound (I) Form B may be performed by forming a supersaturated solution of Compound (I) in the MIBK solvent. Supersaturation may be achieved by, for example, concentrating the solution by removing solvent, cooling the solution or adding a suitable anti-solvent. When crystallisation is initiated by concentrating the solution, the solvent may be removed using well-known methods such as evaporation or distillation. Crystallisation may also be promoted by seeding the solution with Compound (I) Form B crystals. Seeding is particularly advantageous for larger scale preparation of the Compound (I) Form B.

In another aspect, the invention provides a process for the preparation of Compound (I) Form B comprising the following steps:

(i) dissolving Compound (I) in MIBK to form a solution;
(ii) effecting crystallisation of Compound (I) Form B from the solution in step (i); and
(iii) isolating the Compound (I) Form B.

In step (i) the solution of Compound (I) can, for example, be prepared by heating the Compound (I) in the MIBK, suitably to a temperature of 60 to 90° C., such as 60 to 70° C., and particularly at about 85° C. Any form of Compound (I) may be used to prepare the solution in step (i), for example amorphous Compound (I) or Compound (I) Form A.

In step (ii) crystallisation may be effected by, for example distilling off sufficient MIBK to provide a supersaturated solution or by cooling the MIBK to supersaturate the solution. Conveniently however, a proportion of the MIBK is removed by for example distillation or evaporation, followed by cooling. Suitably solvent may be removed by distillation under reduced pressure at a temperature of about 60° C. Generally removal of 40 to 55%, for example 45 to 50% by volume of the solvent is sufficient. Suitably, the mixture is cooled to less than about 10° C., for example about 0 to 10° C., particularly about 2-10° C. In one embodiment the mixture is cooled to about 0 to about −5° C., particularly at about −5° C. The mixture is suitably cooled slowly following the distillation, for example by cooling over a period of a few hours, such as 4 to 5 hours. Following cooling, the mixture may be stirred for a period of time (for example 5 to 20 hours, such as 14 to 18 hours) prior to isolation in step (iii).

In step (iii) the product may be isolated using conventional methods, for example by filtration followed by drying. Drying is suitably performed at a temperature of at 45 to 55° C., conveniently under vacuum.

We have surprisingly found that if the Compound (I) Form B is crystallised from a mixture of MIBK and water, the resulting Compound (I) Form B is produced with low levels of impurities.

Accordingly, in a further aspect of the invention there is provided a process comprising crystallisation of Compound (I) Form B from a mixture of MIBK and water. Suitably in this embodiment Compound (I) is dissolved in a mixture of MIBK and water at elevated temperature, for example 55 to 65° C., particularly about 60° C. The MIBK used in this embodiment suitably contains about 5% w/v water with respect to the MIBK. The mixture is then cooled. Cooling suitably takes place slowly over a period of at least 1 hour to a temperature of about 0 to 10° C., particularly about 2-10° C., more particularly at about 5° C. Suitably the mixture is stirred, for a period of time (for example, at least 1 hour) at the lower temperature to effect complete crystallisation of the product. The Compound (I) Form B is then isolated as hereinbefore described or as illustrated in the Examples.

In a further embodiment of the process for preparing Compound (I) Form B, it may be possible to dissolve Compound (I) in a mixture of MIBK and water in step (i) of the process. The Compound (I) Form B may then be crystallised from the MIBK/water mixture and isolated as described in steps (ii) and (iii) above.

The above methods for preparing Compound (I) Form B from MIBK may also be used to recrystallise Compound (I) Form B. Recrystallisation may be useful for purifying, improving the degree of crystallinity and/or improving the morphology of the Compound (I) Form B crystals.

It is to be understood that for the therapeutic uses, methods of treatment and pharmaceutical compositions described herein, reference to “a Form of Compound (I)” includes Compound (I) Form A and Form B. Accordingly in one aspect of the invention “a Form of Compound (I)” refers to Form A as described herein. In another aspect of the invention “a Form of Compound (I)” refers to Form B as described herein.

The Compound (I) Forms described herein have activity as pharmaceuticals, in particular as modulators human neutrophil elastase. Accordingly the Forms may be beneficial in the treatment or prophylaxis of inflammatory diseases and conditions, for example those diseases and conditions listed below.

1. Diseases of the respiratory tract such as obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus.
2. Diseases of bone and joints including: arthritides associated with or including osteoarthritis/osteoarthrosis, both primary and secondary to, for example, congenital hip dysplasia; cervical and lumbar spondylitis, and low back and neck pain; rheumatoid arthritis and Still's disease; seronegative spondyloarthropathies including ankylosing spondylitis, psoriatic arthritis, reactive arthritis and undifferentiated spondarthropathy; septic arthritis and other infection-related arthropathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositis and polymyositis; polymyalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthralgias, tendonititides, and myopathies.
3. Pain and connective tissue remodelling of musculoskeletal disorders due to injury [for example, sports injury] or disease including: arthritides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritis, scleroderma, mixed connective tissue disorder, spondyloarthropathies or periodontal disease (such as periodontitis).
4. Diseases of skin including: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosis et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia areata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions.
5. Diseases of the eye including: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial.
6. Diseases of the gastrointestinal tract including: glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, non-inflammatory diarrhoea, and food-related allergies which may have effects remote from the gut (for example, migraine, rhinitis or eczema).
7. Diseases of the cardiovascular system including: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins.
8. Oncology including: the treatment of common cancers including prostate, breast, lung, ovarian, pancreatic, bowel and colon, stomach, skin and brain tumours and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes.

Thus, the present invention provides a Form of Compound (I) as hereinbefore defined for use in therapy.

In a further aspect, the present invention provides the use of a Form of Compound (I) as hereinbefore defined in the manufacture of a medicament for use in therapy.

In a further aspect, the present invention provides a Form of Compound (I) as hereinbefore defined for use in the treatment of human diseases or conditions in which modulation of neutrophil elastase activity is beneficial.

In a further aspect, the present invention provides the use of a Form of Compound (I) as hereinbefore defined in the manufacture of a medicament for the treatment of human diseases or conditions in which modulation of neutrophil elastase activity is beneficial.

In a further aspect, the present invention provides a Form of Compound (I) as hereinbefore defined for use in the treatment of an inflammatory disease or condition.

In a further aspect, the present invention provides the use of a Form of Compound (I) as hereinbefore defined in the manufacture of a medicament for the treatment of an inflammatory disease or condition.

In a further aspect, the present invention provides a Form of Compound (I) as hereinbefore defined for use in treating adult respiratory distress syndrome (ARDS), cystic fibrosis, pulmonary emphysema, bronchitis including chronic bronchitis, bronchiectasis, chronic obstructive pulmonary disease (COPD), pulmonary hypertension, asthma including refractive asthma, rhinitis, psoriasis, ischemia-reperfusion injury, rheumatoid arthritis, osteoarthritis, systemic inflammatory response syndrome (SIRS), chronic wound, cancer, atherosclerosis, peptic ulcers, Crohn's disease, ulcerative colitis or gastric mucosal injury.

In a further aspect, the present invention provides the use of a Form of Compound (I) as hereinbefore defined in the manufacture of a medicament for use in treating adult respiratory distress syndrome (ARDS), cystic fibrosis, pulmonary emphysema, bronchitis including chronic bronchitis, bronchiectasis, chronic obstructive pulmonary disease (COPD), pulmonary hypertension, asthma including refractive asthma, rhinitis, psoriasis, ischemia-reperfusion injury, rheumatoid arthritis, osteoarthritis, systemic inflammatory response syndrome (SIRS), chronic wound, cancer, atherosclerosis, peptic ulcers, Crohn's disease, ulcerative colitis or gastric mucosal injury.

In one aspect of the invention the Forms of Compound (I) may be used in the treatment of chronic obstructive pulmonary disease (COPD), cystic fibrosis, bronchiectasis, asthma and rhinitis.

In one aspect, a Form of Compound (I) may be used in the treatment of chronic obstructive pulmonary disease (COPD).

In one aspect, the Forms of Compound (I) may be used in the treatment of cystic fibrosis.

In one aspect, the Forms of Compound (I) may be used in the treatment of bronchiectasis.

Thus, the invention provides the use of a Form of Compound (I) in the manufacture of a medicament for the treatment or prophylaxis of chronic obstructive pulmonary disease (COPD).

In another aspect of the invention there is provided a method of treatment of chronic obstructive pulmonary disease (COPD) comprising administering to a patient in need thereof a therapeutically effective amount of a Forms of Compound (I).

In another aspect of the invention there is provided a method of treating bronchiectasis comprising administering to a patient in need thereof a therapeutically effective amount of a Form of Compound (I) described herein.

In another aspect of the invention there is provided a method of treating cystic fibrosis comprising administering to a patient in need thereof a therapeutically effective amount of a Form of Compound (I) described herein.

Thus, the invention provides the use of a Form of Compound (I) in the manufacture of a medicament for the treatment of cystic fibrosis.

Thus, the invention provides the use of a Form of Compound (I) in the manufacture of a medicament for the treatments of bronchiectasis.

Thus, the invention provides a Form of Compound (I), for use in the treatment of COPD.

Thus, the invention provides a Form of Compound (I), for use in the treatment of cystic fibrosis.

Thus, the invention provides a Form of Compound (I), for use in the treatment of bronchiectasis.

The Forms of Compound (I) described herein may be particularly suitable for use in the treatment of COPD, including the treatment or prophylaxis of symptoms of COPD. Such symptoms include one or more of, dyspnea (breathlessness or shortness of breath), decreased exercise capacity, chronic cough, wheezing or excessive sputum production.

Accordingly, in another aspect of the invention there is provided a method for the reduction of symptoms of COPD (including chronic bronchitis and emphysema) in a patient, comprising administering to a patient in need thereof a therapeutically effective amount of a Form of Compound (I) described herein.

Patients with COPD often experience exacerbations of the condition, resulting in an acute increase in disease symptoms. Such exacerbations are often caused by infection of the tracheobronchial tree or air pollution, however, in many patients the cause of exacerbations is unknown. Exacerbations are a poor prognostic factor for disease progression and patients with exacerbations often require hospitalisation. Exacerbations can result in a permanent reduction in lung function and a worsening of symptoms. There is therefore a need to find suitable methods for preventing or treating such exacerbations. A Form of Compound (I) described herein, may be useful for the treatment of COPD exacerbations. Accordingly a Form of Compound (I), may be useful for treating the severity, frequency and/or duration of COPD exacerbations.

Accordingly, in another aspect of the invention there is provided a method for the reduction of severity, frequency and/or duration of exacerbations in a patient with COPD (including chronic bronchitis and emphysema) comprising administering to a patient in need thereof a therapeutically effective amount of a Form of Compound (I) described herein.

A Form of Compound (I) described herein, may also be useful in stabilising or slowing down disease progression of COPD and may provide a disease modifying effect on COPD. Such disease modification may provide a sustained improvement in lung function and/or lung structure.

According to a further aspect of the invention there is provided a Form of Compound (I) for use in the treatment of AATD.

In one embodiment there is provided a Form of Compound (I), as hereinbefore defined, for use in the treatment of a lung disease (for example COPD or emphysema) in a patient with AATD.

According to another aspect of the invention there is provided the use of a Form of Compound (I) as hereinbefore defined in the manufacture of a medicament for use in the treatment of AATD.

According to another aspect of the invention there is provided the use of a Form of Compound (I) as hereinbefore defined in the manufacture of a medicament for use in the treatment of a lung disease (for example COPD or emphysema) in a patient with AATD.

Patients with AATD may be identified using known methods, for example as described in the minutes of the FDA Advisory Committee on Blood Products 95^{th} Meeting, Jul. 20-21, 2009 and American Thoracic Society/ERS Statement: Standards for the Diagnosis and Management of Individuals with Alpha-1 Antitrypsin Deficiency, Am. J. Respir. Crit. Care Med. 2003; 168:820-899. Diagnosis could include, for example the detection of low serum levels of alpha-1 antitrypsin using conventional methods such as a suitable immunoassay. Currently a serum level below 11 μM (80 mg/dL) is considered to be indicative of AATD, although there is debate about the accuracy of a serum level to determine that a patient has AATD as serum levels can vary between patients. A more accurate method may be to use a genotype test to detect identify alpha-1-antitrypsin deficient alleles, particularly the PI*SZ and PI*ZZ alleles. Patients that are homozygous (PI*ZZ) are expected to be particularly prone to developing conditions such as emphysema or COPD. However heterozygous patients with the PI*Z allele may also be prone to such conditions. Alternatively a phenotype test could be used to determine the specific alpha-1-antitrypsin in a patient. Diagnostic testing could be carried out on a patient without symptoms of a disease. Treatment of such patients may be used to identify patients with AATD and then treat those patients to prevent or delay the onset of conditions such as COPD, emphysema or bronchitis. Alternatively, testing for AATD may be carried out on patients showing symptoms of a disease or condition such as COPD, emphysema or bronchitis.

In one embodiment there is provided a Form of Compound (I) for use in the treatment of a lung disease (for example COPD, emphysema or bronchitis) in a patient diagnosed with AATD. In this embodiment the patient may be diagnosed using, for example, one of the methods described hereinbefore.

In the context of the present specification, the term “therapy” also includes “prophylaxis” unless there are specific indications to the contrary. The terms “therapeutic”, “therapeutically” and “treatment” should be construed accordingly.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

The invention also provides a method of treating, or reducing the risk of, a disease or condition in which inhibition of neutrophil elastase activity is beneficial which comprises administering to a patient in need thereof a therapeutically effective amount of a Form of Compound (I) as hereinbefore defined.

The invention still further provides a method of treating, or reducing the risk of, an inflammatory disease or condition, which comprises administering to a patient in need thereof a therapeutically effective amount of a Form of Compound (I) as hereinbefore defined.

The invention still further provides a method of treating, or reducing the risk of, adult respiratory distress syndrome (ARDS), cystic fibrosis, pulmonary emphysema, bronchitis including chronic bronchitis, bronchiectasis, chronic obstructive pulmonary disease (COPD), pulmonary hypertension, asthma including refractive asthma, rhinitis, psoriasis, ischemia-reperfusion injury, rheumatoid arthritis, osteoarthritis, systemic inflammatory response syndrome (SIRS), chronic wound, cancer, atherosclerosis, peptic ulcers, Crohn's disease, ulcerative colitis or gastric mucosal injury which comprises administering to a patient in need thereof a therapeutically effective amount of a Form of Compound (I) as hereinbefore defined.

The invention still further provides a method of treating, or reducing the risk of developing, chronic obstructive pulmonary disease (COPD) which comprises administering to a patient in need thereof a therapeutically effective amount of a Form of Compound (I) as hereinbefore defined.

The invention still further provides a method of treating, or reducing the risk of, cystic fibrosis, which comprises administering to a patient in need thereof a therapeutically effective amount of a Form of Compound (I) as hereinbefore defined.

The invention still further provides a method of treating, or reducing the risk of developing, bronchiectasis, which comprises administering to a patient in need thereof a therapeutically effective amount of a Form of Compound (I) as hereinbefore defined.

The invention still further provides a method of treating AATD, or reducing the risk of developing a condition associated with AATD, which comprises administering to a patient in need thereof a therapeutically effective amount of a Form of Compound (I) as hereinbefore defined.

The invention still further provides a method of treating AATD, or reducing the risk of developing a condition associated with AATD, which comprises diagnosing a patient with AATD and administering to said patient a therapeutically effective amount of a Form of Compound (I) as hereinbefore defined.

The invention still further provides a method of treating, or reducing the risk of developing, a lung disease such as COPD, emphysema or bronchitis (for example COPD or emphysema) in a patient with AATD, which comprises administering to a patient in need thereof a therapeutically effective amount of a Form of Compound (I) as hereinbefore defined.

The invention still further provides a method of treating, or reducing the risk of developing, a lung disease such as COPD or emphysema in a patient with AATD, which comprises
(i) testing a patient for AATD; and
(ii) when said testing determines that said patient has AATD, administering a therapeutically effective amount of a Form of Compound (I) as hereinbefore defined.

In this embodiment the patient in step (i) may be symptom-free of a lung disease such as COPD, emphysema or bronchitis before being tested for AATD. In this case, the method of treatment may prevent the patient from developing the lung disease, or may prevent or delay progression at an early stage of the lung disease. Alternatively, the patient may have symptoms of a lung disease such as COPD, emphysema or bronchitis, prior to testing for AATD.

In this embodiment the testing/diagnosis of AATD may, for example be carried out as hereinbefore defined.

For the above-mentioned therapeutic uses the dosage administered will, of course, vary with the compound employed, the mode of administration, the treatment desired and the disorder indicated. The daily dosage of Compound (I) may be in the range from 0.001 mg/kg to 100 mg/kg, for example 0.001 to 1 mg/kg, suitably 0.001 to 1 mg/kg or 0.001 to 0.2 mg/kg and particularly 0.001 to 0.01 mg/kg.

### Pharmaceutical Compositions

The Forms of Compound (I) as hereinbefore defined may be used on their own but will generally be administered in the form of a pharmaceutical composition in which the Form of Compound (I) (active ingredient) is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration, the pharmaceutical composition will preferably comprise from 0.05 to 99% w/w (percent by weight), for example 0.05 to 90% w/w, 0.05 to 80% w/w, 0.10 to 70% w/w, 0.1 to 60% w/w, 0.1 to 50% w/w 0.1 to 40% w/w, 0.1 to 30% w/w, 0.1 to 20% w/w or 0.1 to 5% w/w of active ingredient, all percentages by weight being based on total composition.

The present invention also provides a pharmaceutical composition comprising a Form of Compound (I) as hereinbefore defined, in association with a pharmaceutically acceptable adjuvant, diluent or carrier.

The invention further provides a process for the preparation of a pharmaceutical composition of the invention which comprises mixing a Form of Compound (I) as hereinbefore defined, with a pharmaceutically acceptable adjuvant, diluent or carrier.

The pharmaceutical compositions may be administered topically (e.g. to the skin or to the lung and/or airways (including the nasal cavity)) in the form, e.g., of creams, solutions, suspensions, heptafluoroalkane (HFA) aerosols and dry powder formulations, for example, formulations in the inhaler device known as Turbuhaler®; or systemically, e.g. by oral administration in the form of tablets, capsules, syrups, suspensions, solutions, powders or granules; or by parenteral administration in the form of solutions or suspensions; or by subcutaneous administration; or by rectal administration in the form of suppositories; or transdermally.

### Pharmaceutical Compositions for Administration by Inhalation

In a particular embodiment of the invention the Forms of Compound (I) as hereinbefore defined are administered by inhalation (oral or nasal) for the treatment of respiratory diseases, for example as herein described, such as chronic obstructive pulmonary disease (COPD) or asthma. When administered by inhalation the Forms of Compound (I) as hereinbefore defined may be used effectively at unit doses in the μg/kg range, for example 0.1 to 500 μg/kg, 0.1 to 250 μg/kg, 0.1 to 100 μg/kg, 0.1 to 50 μg/kg, 0.1 to 40 μg/kg, 0.1 to 30 μg/kg, 0.1 to 20 μg/kg, 0.1 to 10 μg/kg, 5 to 500 μg/kg, 5 to 250 μg/kg, 5 to 100 μg/kg, 5 to 10 μg/kg, 5 to 50 μg/kg, 5 to 40 μg/kg, 5 to 30 μg/kg, 5 to 20 μg/kg, 10 to 500 μg/kg, 10 to 250 μg/kg, 10 to 100 μg/kg, 10 to 50 μg/kg, 10 to 40 μg/kg 10 to 30 μg/kg, or 10 to 20 μg/kg of active ingredient. For example, a daily dose of about 6 μg/kg or about 12 μg/kg of active ingredient. The total daily dose may be administered as a single dose or as multiple doses per day, for example twice daily dosing.

In an embodiment of the invention, there is provided a pharmaceutical composition comprising a Form of Compound (I) as hereinbefore defined (for example Form B), in association with a pharmaceutically acceptable adjuvant, diluent or carrier, which is formulated for inhaled administration (including oral and nasal inhalation).

When administered by inhalation, metered dose inhaler devices may be used to administer the Form of Compound (I), dispersed in a suitable propellant and with or without additional excipients such as ethanol, surfactants, lubricants or stabilising agents. Suitable propellants include hydrocarbons, chlorofluorocarbons and hydrofluoroalkanes (e.g. heptafluoroalkane) propellants, or mixtures of any such propellants. Preferred propellants are P134a and P227, each of which may be used alone or in combination with other propellants and/or surfactant and/or other excipients. Nebulised aqueous suspensions or, preferably, solutions may also be employed, with or without a suitable pH and/or tonicity adjustment, either as a unit-dose or multi-dose formulations. For example a suitable composition for inhalation as a nebulised suspension comprises the Form of Compound (I) (typically at a concentration of about 1 to 20 mg/g) dispersed in an aqueous medium (mg/g in Mill-Q water) comprising sodium chloride (9 mg/g); citric acid dried (0.0735 mg/g); sodium citrate (0.19 mg/g); benzalkonium chloride (0.1 mg/g), EDTA (ethylenediamine tetraacetic acid, 0.1 mg/g) and Polysorbate 80 (0.3 mg/g).

Dry powder inhalers may be used to administer the active ingredient, alone or in combination with a pharmaceutically acceptable carrier, in the later case either as a finely divided powder, as an agglomerated/spheronized mixture, or as an ordered mixture. The dry powder inhaler may be single dose or multi-dose and may utilise a dry powder reservoir or a powder-containing capsule or blister.

Metered dose inhaler, nebuliser and dry powder inhaler devices are well known and a variety of such devices are available.

In a further embodiment, the pharmaceutical composition is administered by means of a dry powder inhaler (DPI).

The DPI may be “passive” or breath-actuated, or “active” where the powder is dispersed by some mechanism other than the patient's inhalation, for instance, an internal supply of compressed air. At present, three types of passive dry powder inhalers are available: single-dose, multiple unit dose or multidose (reservoir) inhalers. In single-dose devices, individual doses are provided, usually in gelatine capsules, and have to be loaded into the inhaler before use, examples of which include Spinhaler® (Aventis), Rotahaler®(GlaxoSmithKline), Aeroliser™ (Novartis), Inhalator® (Boehringer) and Eclipse (Aventis) devices. Multiple unit dose inhalers contain a number of individually packaged doses, either as multiple gelatine capsules or in blisters, examples of which include Diskhaler® (GlaxoSmithKline), Diskus® (GlaxoSmithKline) and Aerohaler® (Boehringer) devices. In multidose devices, drug is stored in a bulk powder reservoir from which individual doses are metered, examples of which include Turbuhaler® (AstraZeneca), Easyhaler® (Orion), Novolizer® (ASTA Medica), Clickhaler® (Innovata Biomed) and Pulvinal® (Chiesi) devices.

An inhalable pharmaceutical composition or dry powder formulation for use in a DPI can be prepared by mixing finely divided active ingredient (having a mass median diameter generally equal to or less than 10 μm, preferably equal to or less than 5 μm, for example from 1 to 5 μm) with a carrier substance, for example, a mono-, di- or polysaccharide, a sugar alcohol, or another polyol. Suitable carriers are sugars or sugar alcohols, for example, lactose, glucose, raffinose, melezitose, lactitol, maltitol, trehalose, sucrose, mannitol; and starch.

The Form of Compound (I) may be prepared as finely divided particles using well known size reduction methods such as milling. Suitably the Form is micronised by charging the substance continuously to a jet mill by a screw feeder at a feed rate of, for example 2 to 8 kg/hour, depending on the size of the mill. The outer chamber pressure of the mill is controlled at about 2 to 6 bar and the ejector pressure adjusted relative to the chamber pressure so as to prevent blow back of material from the mill.

The preparation of finely divided active and/or carrier materials, for example by milling, can result in damage to the crystalline structure of the active/carrier materials. The crystallinity of the particles may be restored using known methods. For example, analogous conditioning processes to those described in WO92/18110 and WO 95/05805 may be used with the Forms of Compound (I) described herein. Suitably the Form of Compound (I) may be conditioned in a mixture of water and ethanol vapour, for example a conditioning in ethanol vapor with an activity of 0.7 (70% saturated ethanol vapour). The conditioning vapour may be prepared using known methods such as preparing a saturated ethanol solution with sodium iodide; passing nitrogen (or air) pass through ethanol at a specific temperature and thereafter diluting the gas stream with pure nitrogen to the give the required ethanol concentration; or preparing saturated ethanol vapor at a specific temperature and then increasing the temperature to obtain the desired ethanol activity. The conditioning is suitably performed at about 25° C. for about 20 hours. The particles of the Form may be conditioned alone or in admixture with particles of a carrier such as lactose monohydrate.

If required the dry powder composition may contain a suitable coating agent such as magnesium stearate, ascorbyl palmitate or sodium stearyl fumarate. Alternatively the Form of Compound (I) may be used alone in a DPI. The powder mixture (or Form of compound (I) alone) may then, as required, be dispensed into hard gelatine capsules or blisters, each containing the desired dose of the active ingredient.

In embodiments, the particles of the active ingredient adhere to the carrier particles to form an ordered (interactive) powder mixture. The carrier particles may have a mass median diameter of from 20 to 1000 μm, more usually from 50 to 500 μm.

Alternatively, an inhalable pharmaceutical composition may be prepared by processing a finely divided powder (e.g. consisting of finely divided active ingredient and finely divided carrier particles) into spheres that break up during the inhalation procedure. Examples of suitable spheronized active/carrier powders include analogous products to those described in WO 98/031350, WO 98/031351 or WO 98/031352. The spheronized powder mixtures may be prepared using known methods, for example by preparing a micronized homogenous mixture of the Form and a carrier such as lactose monohydrate, and spheronizing the mixture as described in WO 95/09615.

The spheronized powder is filled into the drug reservoir of a multidose inhaler, for example, that known as the Turbuhaler® in which a dosing unit meters the desired dose which is then inhaled by the patient.

In a further embodiment, the pharmaceutical composition is administered by means of a metered dose inhaler, particularly a pressurised metered dose inhaler (pMDI). The pMDI contains the active as a suitable solution or suspension in a pressurised container. The active is delivered by actuating a valve on the pMDI device. Actuation may be manual or breath actuated. In manually actuated pMDIs the device is actuated by for example pressing a suitable release mechanism on the pMDI device as the patient inhales. Breath actuated pMDIs are actuated when the patient inhales through the mouthpiece of the pMDI. This can be advantageous as the actuation of the device is timed with the patients' inhalation and can result in a more consistent dosing of the active. Examples of pMDI devices include for example Rapihaler® (AstraZeneca), Vannair® (AstraZeneca), Ventolin® HFA, Evohaler® (GlaxoSmithKline), Maxair® Autohaler® (Graceway Pharmaceuticals), Easi-Breathe® (IVAX International).

In a further embodiment, the Compound (I) is administered by means of a metered dose inhaler in combination with a spacer. Suitable spacers are well known and include Nebuchamber® (AstraZeneca) or Volumatic® (GlaxoSmithKline).

In a further embodiment, the Form of Compound (I) is administered by means of a nebuliser. Suitable nebulisers are well known and include the eFlow® (PARI GmbH).

In a further embodiment, the Form of Compound (I) is administered by means of a metered dose liquid inhaler (MDLI) or a small volume nebuliser (SVN). The MDLI or SVN contains the active in a solution or suspension in a reservoir. The formulation of the suspension or solution may contain just the active, or may contain additional excipients such as solvents surfactants, lubricants or stabilising agents. Means for dispensing the formulation are provided in communication with the reservoir, in particular a mesh or membrane that is vibrated by a piezoelectric element to form fine droplets of liquid that are dispensed into the lung or nasal cavity.

An inhalable pharmaceutical composition for use in a nebuliser or MDLI can be prepared by dispersing or preferably dissolving the Form of Compound (I) in a suitable aqueous medium. The composition may also include for example suitable pH and/or tonicity adjustment, surfactants and preservatives.

When administered intra-nasally, the Form of Compound (I) could be administered as a solution, or a suspension in a suitable aqueous medium for a suitable nasal delivery device such as a spray pump or a pMDI, for example Rhinocort Aqua® (AstraZeneca). Alternatively the compound could be administered as a dry powder composition as hereinbefore described using a suitable DPI device e.g. Rhinocort® or Turbuhaler® (AstraZeneca). If it is desirable to keep the compound in the nasal region it may be necessary to use a larger particle size in the dry powder composition, for example greater than 10 μm, such as 10 to 50 μm.

Accordingly, the present invention also provides an inhaler device (for example a dry powder inhaler, in particular a multiple unit dose dry powder inhaler, or a pMDI inhaler) containing an inhalable pharmaceutical composition of the invention.

The invention further relates to combination therapies wherein a Form of Compound (I) according to the invention, or a pharmaceutical composition comprising such a Form, is administered concurrently or sequentially or as a combined preparation with another therapeutic agent or agents, for the treatment of one or more of the conditions listed. Examples of other therapeutic agent or agents which could be used in combination with the Form of Compound (I) include the therapeutic agents disclosed in WO2007/129963, incorporated herein by reference thereto.

For example a Form of Compound (I) may be administered concurrently or sequentially or as a combined preparation with another therapeutic agent or agents selected from:
a) a PDE4 inhibitor including an inhibitor of the isoform PDE4D;
b) a β-adrenoceptor agonist such as metaproterenol, isoproterenol, isoprenaline, albuterol, salbutamol, formoterol, salmeterol, terbutaline, orciprenaline, bitolterol mesylate, pirbuterol or indacaterol;
c) a muscarinic receptor antagonist (for example a M1, M2 or M3 antagonist, such as a selective M3 antagonist) such as ipratropium bromide, tiotropium bromide, oxitropium bromide, pirenzepine or telenzepine;
d) a modulator of chemokine receptor function (such as a CCR1 or CCR8 receptor antagonist);
e) an inhibitor of kinase function;
f) a non-steroidal glucocorticoid receptor agonist;
g) a steroidal glucocorticoid receptor agonist; and
h) a protease inhibitor (such as a MMP12 or MMP9 inhibitor);

### Preparation of Compound (I)

Compound (I), or a pharmaceutically acceptable salt thereof and certain intermediates useful in the synthesis thereof may be prepared using the methods described in WO2007/129963 and WO2009/061271. Compound (I) may also be prepared using the method described in the Examples herein.

As a further aspect of the present invention there is provided a process for the preparation of Compound (I) comprising the reaction of a compound of the formula (II), or an activated derivative thereof:

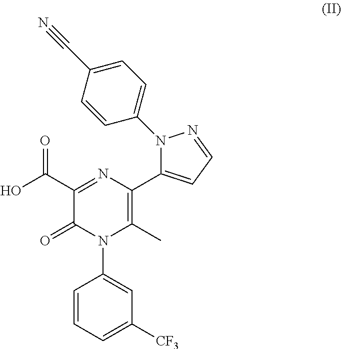

with ethylamine.

Suitable activated derivatives of the compound of formula (II) are carboxylic acid derivatives of the compound of formula (II) suitable for amide formation. Such reactive derivatives could include for example, an acyl halide, for example an acyl chloride formed by the reaction of the acid with an inorganic acid chloride, for example thionyl chloride; a mixed anhydride, for example an anhydride formed by the reaction of the acid with a chloroformate such as isobutyl chloroformate; an active ester, for example an ester formed by the reaction of the acid with a phenol such as pentafluorophenol, or with an alcohol such as methanol, ethanol, isopropanol, butanol or N-hydroxybenzotriazole; an acyl azide, for example an azide formed by the reaction of the acid with an azide such as diphenylphosphoryl azide; an acyl cyanide, for example a cyanide formed by the reaction of an acid with a cyanide such as diethylphosphoryl cyanide; or the product of the reaction of the acid with a carbodiimide such as dicyclohexylcarbodiimide, with 1,1′-carbonyl diimidazole, or with a uronium compound such as 2-(7-azabenzotriazol-1-yl)-1,1,3,3-tetramethyluronium hexafluorophosphate(V). A particular activated derivative is an alkyl ester, for example the methyl ester, of the compound of formula (II).

The reaction is conveniently carried out in a suitable solvent or diluent. For example, when the compound of the formula (II) is used in the form of an alkyl ester, such as the methyl ester, the reaction is conveniently carried out in for example, an alcohol such as methanol, ethanol or isopropanol. Alternatively a mixture of solvents may be used such as a mixture of methanol, acetonitrile and water.

When the compound of the formula (II) is used in the form of an acyl halide such as the acyl chloride the reaction is suitably carried out in, for example, dichloromethane, tetrahydrofuran, methyl tert-butyl ether, toluene or N,N-dimethylformamide.

The reaction is conveniently carried out at a temperature in the range, for example, 0 to 120° C., preferably at or near ambient temperature.

The compound of formula (II) may be prepared for example by cross-coupling a compound of the formula (III):

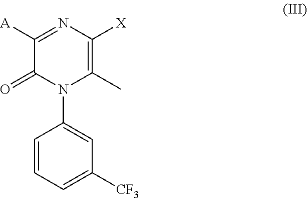

wherein A is a carboxy group or a suitable activated derivative thereof; and X is halo (for example chloro, bromo or iodo) or a triflate group (trifluoromethanesulfonate);

with a compound of the formula (IV):

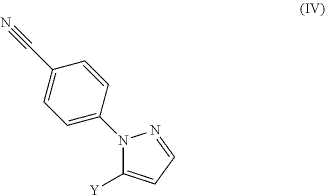

wherein Y is a boronic acid or an ester thereof, a trifluoroborate group or a suitable zincate.

When A in the compound of formula (III) is a suitable activated derivative of the carboxy group, it is a reactive derivative suitable for the formation of the amide described above, and which is sufficiently stable to survive the conditions used in the cross-coupling reaction. For example A is an ester such as the methyl or ethyl ester or A is carboxy. In one embodiment A is MeOC(O)—.

The group Y in the compound of formula IV is a boronic acid group (B(OH)₂) or an ester thereof, a trifluoroborate group or a suitable zincate. When Y is a zincate the coupling reaction may be performed as a Negishi reaction, using analogous conditions to those described in, for example J. Am. Chem. Soc., 2004, 126 (40), pp 13028-13032. Suitable zincates include for example, those where Y in the compound of formula IV is ZnX¹ and X¹ is chloro, bromo or iodo. When Y is a trifluoroborate group it is a suitable salt, for example potassium trifluoroborate. Examples of boronic acid esters represented by Y include alkyl esters, stabilised esters, for example a N-methyliminodiacetic acid boronate (such as the MIDA boronates described in J. Am. Chem. Soc., 2009, 131, 6961) or the pinacol ester. A particular compound of the formula (IV) is the compound of the formula (IVa):

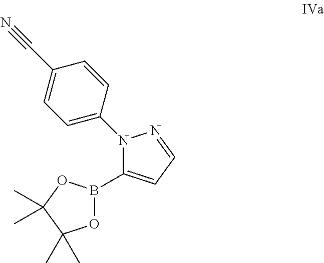

The coupling reaction is performed in the presence of a suitable base, for example an inorganic or organic base. Suitable inorganic bases include for example, a carbonate such as potassium carbonate, a phosphate such as potassium phosphate dibasic (K₂HPO₃) or potassium phosphate tribasic (K₂PO₄) or a hydroxide base such as barium, sodium or potassium hydroxide. Suitable organic bases include an organic amine such as triethylamine or N-diisopropylethylamine (Hunigs base), or an alkali metal bases such as sodium acetate or a sodium alkoxide such as sodium methoxide or sodium ethoxide.

The reaction is performed in the presence of a suitable palladium catalyst. Suitable catalysts include, for example, palladium with suitable ligands, typically organo-phosphorus ligands. Conveniently the palladium catalyst is generated in-situ in the reaction mixture by reacting a suitable palladium source, such as palladium (II) acetate or tris(dibenzylideneacetone)dipalladium(0) with the required ligand. Examples of ligands that may be used to generate the catalyst include a ligand selected from 2-dicyclohexylphosphino-2′,6′-dimethoxy-1,1′-biphenyl; tri-tert-butylphosphine, triphenylphosphine; tri-(4-fluorophenyl)phosphine; tri-(2-furyl)phosphine; 1-phenyl-2,2,6,6-tetramethylphosphacyclohexan-4-one; phenyldi(tert-butyl)phosphine; tert-butylphenylphosphine; 4,5-bis(diphenylphosphino)-9,9-dimethylxanthene; 4,6-bis(diphenylphosphino)phenoxazine, 1,3,5,7-tetramethyl-6-phenyl-2,4,8-trioxa-6-phosphaadamantane, tricyclohexylphosphine; tri-o-tolylphosphine; Di(1-adamantyl)-n-butylphosphine; 1,3-dihydro-1,3-bis(2,4,6-trimethylphenyl)-2H-imidazol-2-ylidene; 1,3-bis[2,6-bis(1-methylethyl)phenyl]-1,3-dihydro-2H-imidazol-2-ylidene; 1,1′-Bis(di-tert-butylphosphino)ferrocene; 1,1′-Bis(di-iso-propylphosphino)ferrocene; and 1,2-Bis(diphenylphosphino)ethane.

A particular example of a suitable palladium catalyst is dichloro 1,1-bis(di-tert-butylphosphino)ferrocene palladium (II) dichloride.

The reaction is carried out in the presence of water, for example 1 mole equivalent or 50% v/v as solvent. In addition to the water the reaction is conveniently performed in a suitable solvent, for example dimethylformamide, 2-methyl-tetrahydrofuran, acetonitrile, 1-methyl 2-pyrrolidinone, dimethoxyethane, dioxane, toluene, anisole, an alcohol (for example, ethanol or isopropanol), a ketone (for example, 4-methylpentan-2-one (methyl isobutyl ketone—MIBK) or an ester (for example, butyl acetate). The reaction is suitably performed at ambient or elevated temperature, for example at about 20° C. or at about 50-90° C., for example about 80° C.

When A in the compound of formula (III) is carboxy the reaction results in the formation of compound (II). If required the compound of formula (II) may be activated as hereinbefore described prior to the subsequent reaction with ethylamine to give Compound (I).

The compounds of formulae (III) and (IV) may be prepared, for example, using the methods described in the Examples herein.

Compound (I) could also be prepared by coupling a compound of the formula (V):

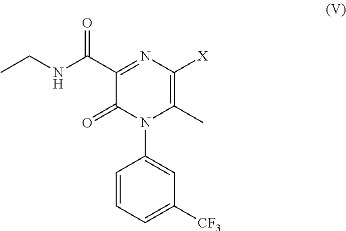

wherein X is as hereinbefore defined;
with a compound of the formula (IV) as hereinbefore defined.

This reaction could be performed using analogous conditions to those described for the coupling of the compound of formulae (III) and (IV) described above.

The compound of formula (V) could be prepared using analogous methods to those described in WO2007/129963.

Accordingly Compound (I) From B could be prepared by a process comprising:
(i) the reaction of a compound of the formula (V) as hereinbefore defined with a compound of the formula (IV) as hereinbefore defined; and
(ii) preparing Compound (I) Form B using any of the methods described herein, for example by crystallising Form B from a suitable solvent (such as MIBK) as hereinbefore defined.

According to the further aspect of the invention there is provided a process for the preparation of Compound (I) comprising:

(i) the coupling of a compound of the formula (III) with a compound of formula (IV) to give the compound of the formula (II), or an activated derivative thereof as hereinbefore defined; and

(ii) the reaction of the compound of the formula (II), or an activated derivative thereof with ethylamine as hereinbefore defined.

According to the further aspect of the invention there is provided a process for the preparation of Compound (I) Form B comprising:

(i) the reaction of the compound of the formula (II), or an activated derivative thereof with ethylamine as herein before defined to give Compound (I); and

(ii) preparing Compound (I) Form B using any of the methods described herein, for example by crystallising Form B from a suitable solvent (such as MIBK) as hereinbefore defined.

According to the further aspect of the invention there is provided a process for the preparation of Compound (I) Form B comprising:

(i) the coupling of a compound of the formula (III) with a compound of formula (IV) to give the compound of the formula (II), or an activated derivative thereof as hereinbefore defined;

(ii) the reaction of the compound of the formula (II), or an activated derivative thereof with ethylamine as hereinbefore defined to give Compound (I); and

(iii) preparing Compound (I) Form B using any of the methods described herein, for example by crystallising Form B from a suitable solvent (such as MIBK) as hereinbefore defined. Suitably, in the processes described above, the reaction of the compound of formula (II) with ethylamine can provide Compound (I) Form A, by crystallising the Form A from a suitable solvent, such as methanol, or from a mixture of methanol, acetonitrile and water. If required, the Form A may then converted to Form B using one of the methods described herein for preparing From B, such as recrystallisation from MIBK.

### EXAMPLES

The invention will now be illustrated by the following Examples in which, unless stated otherwise:

(i) temperatures are given in degrees Celsius (° C.); operations were carried out at room or ambient temperature, that is, at a temperature in the range of 18-25° C. and under an atmosphere of an inert gas such as argon.
(ii) Unless stated otherwise, the NMR spectra were recorded on a Varian Unity-Inova 500 MHz instrument using TMS as an internal standard in CDCl₃ solvent.
(iii) In general, the course of reactions was followed by HPLC and reaction times are given for illustration only.
(iv) Yields are given for illustration only and are not necessarily those which can be obtained by diligent process development; preparations were repeated if more material was required.
(v) Chemical symbols have their usual meanings; SI units and symbols are used.
(vi) Solvent ratios are given in volume:volume (v/v) terms.
(vi) Unless stated otherwise, starting materials were commercially available.

### X-Ray Powder Diffraction (XPRD)

Ambient X-Ray Powder Diffraction (XRPD) patterns on Compound (I) Form B were collected on a PANalytical X'Pert PRO MPD theta-theta system using nickel-filtered CuK-radiation (1.5418 Å, 45 kV, 40 mA) and an X'Celerator detector. A programmable divergence slit and a programmable anti-scatter slit giving a constant irradiated length of 10 mm was used. The diffraction patterns were collected between 2 and 40° 2θ in a continuous scan mode. The scan speed was 4°/min, with an increment of 0.016°. Thin flat samples were prepared on flat silicon zero background plates. The plates were mounted in a sample holder and rotated in a horizontal position during measurement.

The X-ray Powder Diffraction patterns on Compound (I) Form A measured under controlled in humidity and temperature, were collected on a PANalytical X'Pert PRO MPD theta-theta system, equipped with a focusing beam Johansson monochromator and a X'Celerator detector, using CuKα1 radiation (1.5406 Å, 45 kV, 40 mA). The diffraction pattern was collected between 2 and 40° 2θ in a continuous scan mode. The scan speed was 0.86°/min, with an increment of 0.016°. To obtain a controlled atmosphere, an Anton Paar THC chamber was mounted and used in the diffractometer. The humidity was generated and controlled by a VTI RH-200, which feeds a humidified nitrogen gas into the Anton Paar THC chamber. The temperature of the sample was controlled with an Anton Paar TCU 50 temperature control unit. A thin sample was prepared on a flat holder, provided with the Anton Paar THC chamber. No spinning of the sample was performed during the measurement.

### Humidity Interaction

The gravimetric responses of test samples to changes in humidity were investigated using a TGA 5000 (TA Instruments) Gravimetrical Vapour Sorption (GVS). The temperature was held at 25° C. throughout the experiments. The relative humidity (RH) was raised in steps (of 5 or 10%) up to 90% RH and lowered back to 0% RH in two cycles. Each level of RH was held until the equilibrium condition (sample weight change<0.0005 wt % per 10 minutes) was reached. 5-10 mg of the test sample was placed in the cup and evaluated. The hygroscopicity was calculated as the relative change in weight of the sample between 0% RH at the start of the second cycle and 80% RH during the increase of humidity in the second cycle.

The hygroscopicity of a sample is dependent on factors in addition to the inherent properties of the pure solid form itself, for example the purity and the crystallinity of the sample will have some impact on the result.

### Differential Scanning Calorimetry (DSC)

Using standard methods (for example those described in Höhne, G. W. H. et al (1996), Differential Scanning calorimetry, Springer, Berlin) the calorimetric response of a test sample to increasing temperature was investigated using a TA Instruments Q2000 Modulated Temperature Differential Scanning calorimeter (MTDSC). Measurements were performed between 15 and 250° C. using a modulation of ±0.50° C. in intervals of 40 seconds and a ramp rate of 5° C. per minute. Approximately 1 to 5 mg of test sample was placed in aluminium cups with lids (no crimping) under a nitrogen atmosphere.

As mentioned hereinbefore, it is well known that the DSC onset and peak temperatures may vary according to the purity of the sample and instrumental parameters, especially the temperature scan rate. A person skilled in the art can use routine optimization/calibration to set up instrumental parameters for a differential scanning calorimeter so that data comparable to the data presented here can be collected.

### ABBREVIATIONS

### DME: Dimethoxyethane

### DMF: N,N-dimethylformamide

EtOAc: Ethyl acetate
LOD: Loss on drying

### MeOH: Methanol

MIBK: Methyl isobutyl ketone
MTBE: Methyl t-butyl ether
NMP: 1-Methyl 2-pyrrolidinone

### THF: Tetrahydrofuran

eq: equivalents
mins: minutes
h: hours

### Example 1

### Preparation of Compound (I) Form A

Compound (I) (45 mg) was dissolved in ethanol (0.5 mL) and stirred over night. After 24 hours precipitated solid material was isolated by filtration and washed with 0.5 mL of ethanol and allowed to dry in air, to give compound (I) Form A, yield 38 mg (84%). The solid form was analysed with X-ray powder diffraction, which confirmed that the Compound (I) Form A was crystalline.

### Example 2

### Preparation of Compound (I) Form A

Methyl 6-(1-(4-cyanophenyl)-1H-pyrazol-5-yl)-5-methyl-3-oxo-4-(3-(trifluoromethyl)phenyl)-3,4-dihydropyrazine-2-carboxylate (15.00 g, 31.29 mmol) and ethylamine 2M in methanol (62.6 ml, 125.15 mmol) in a 250 mL round bottomed flask was heated to 55° C. for two hours. The reaction mixture was evaporated to dryness affording approximately 16 g of a violet solid. 90% ethanol (100 mL) was added and heated to reflux for 30 minutes, the mixture stirred and allowed to cool to room temperature (approximately 2 hours). The solid was filtered, washed with ethanol and dried. The resulting grey solid was recrystallised from 90% ethanol (100 mL), stirring overnight. Filtered and washed with small amounts of ethanol afforded again a light greyish powder. This was purified by flash chromatography on silica using dichloromethane:methanol as eluents (99:1 to 98:2). The pure fractions were evaporated and afforded a yellow-orange foam 13.8 g, which was recrystallised from ethanol 90% (100 mL). The mixture was stirred overnight, filtered and washed with ethanol, air dried to give an off-white solid (approximately 12.5 g, containing approximately 20-25 mol % ethanol. The solid was then dried under vacuum at 50° C. for more than 20 hours giving 6-[2-(4-cyano-phenyl)-2H-pyrazol-3-yl]-5-methyl-3-oxo-4-(3-trifluoromethyl-phenyl)-3,4-dihydro-pyrazine-2-carboxylic acid ethylamide Form A containing 15 mol % (1.3% by weight) ethanol. The product was micronised and analysed using NMR and found to contain approximately 10 mole % (1.1% by weight) ethanol.

### Summary of Characteristics of Compound (I) Form A.

X-ray powder diffraction analysis of compound (I) Form A gave the XRPD pattern substantially as shown in FIG. 1 when measured under measured under controlled conditions of 5% relative humidity at 25° C. (CuKα1 radiation (1.5406 Å, 45 kV, 40 mA)). The most prominent peaks (2θ value) of Compound (I) Form A are shown in Table 1 hereinbefore.

The hygroscopicity of Compound (I) Form A, defined as the water uptake by a dried sample when the relative humidity is increased from 0% to 80% at 25° C., was 1.6% (w/w).

### Example 3

### Preparation of Compound (I) Form B

Compound (I) Form A (approximately 5 mg) was heated in a DSC pan using a TA Instruments Q2000. The material was heated with a heating rate of 10°/min to 210° C., and held at 210° C. for three minutes. Then the temperature was lowered with a rate of 5° C./min to room temperature to give Compound (I) Form B.

### Example 4

### Preparation of Compound (I) Form B

### 4a: Ethyl 2-oxo-2-(3-(trifluoromethyl)phenylamino)acetate

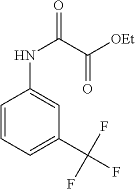

3-(Trifluoromethyl) aniline (500.0 g), triethylamine (439.1 g) and ethyl acetate (5000 mL) were charged into the reactor and cooled to 0-5° C., then ethyl oxalyl chloride (508.7 g) was added dropwise, at the rate of 2-4-drops/s, keeping the reaction temperature at 0-10° C. When the addition was complete, the mixture was stirred for 30 min at 0-10° C. Then the mixture was warmed to 15-25° C. and held for 1-2 hours, then sampled it to be detected until the content of starting material was <1%. Then water (3350 mL) was added slowly at about 15 to 25° C. to quench the reaction. After the addition, the mixture was stirred for another 30 mins. The mixture was separated and the water phase extracted with ethyl acetate (13350 ml). The organic phases were combined and washed with 20% brine (1000 ml) after water (1250 ml), then concentrated under reduced pressure (<50° C., pressure<−0.08 MPa) until the wt % of ethyl acetate was <20%. Then petrol ether (1500 ml) was added to the residue and filtered off. The cake was washed with petrol ether (260 ml) and dried in an oven at <50° C. to afford ethyl 2-oxo-2-(3-(trifluoromethyl)phenylamino)acetate (789.0 g, purity=98.3%, yield=97.3%); ¹H NMR: (500 MHz CDCl₃): 1.40 (t, 3H), 4.42 (q, 2H), 7.45 (d, 1H), 7.51 (t, 1H), 7.87 (s, 1H), 7.92 (s, 1H), 9.00 (s, 1H).

### 4b: N₁-(2-hydroxypropyl)-N₂-(3-(trifluoromethyl)phenyl)oxalamide

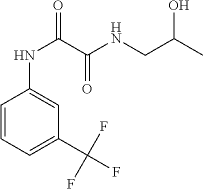

Ethyl 2-oxo-2-(3-(trifluoromethyl)phenylamino)acetate (789.0 g) and ethanol (5129 ml) were charged into a reactor and the mixture was heated to 70-75° C. at the rate of 40-45° C./hour to give a gentle reflux, then 1-amino-2-propanol (249.4 g) was added at the rate of 0.2 g-0.4 g/minute keeping the temperature at 70-75° C. After the addition, the mixture was held for 2-3 hours at 70-75° C. The mixture was then cooled to <50° C. and evaporated ethanol until the wt % of ethanol was <20%. Then heptane (4182 ml) was added. The mixture was cooled to 0-5° C. and stirred for 2-3 hours, then filtered and the cake was washed with (760 ml) heptane, dried in the oven under 50° C. to afford N₁-(2-hydroxypropyl)-N₂-(3-(trifluoromethyl)phenyl)oxalamide (760.0 g, purity=99.9%, yield=87.7%); ¹H NMR: (500 MHz CDCl₃): 1.25 (d, 3H), 2.30 (s, 1H), 3.30 (m, 1H), 3.55 (m, 1H), 4.02 (m, 1H), 7.43 (d, 1H), 7.47 (t, 1H), 7.81 (d, 1H), 7.99 (s, 1H), 8.01 (s, 1H), 9.52 (s, 1H).

### 4c: N₁-(2-oxopropyl)-N₂-(3-(trifluoromethyl)phenyl)oxalamide

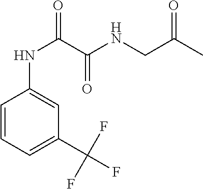

N₁-(2-hydroxypropyl)-N₂-(3-(trifluoromethyl)phenyl)oxalamide (464.0 g), acetonitrile (6590 ml) and ruthenium chloride hydrate (4.8 g) in water (561 ml) were charged to the reactor and kept the contents at 20-25° C. Then sodium bromate (265.8 g) solution in water (114 ml) was added to the reactor. After the addition, the mixture was held at 20-25° C. for 2-4 hours. Water (9280 ml) was added to the mixture and stirred at 10° C.-20° C. for 2-3 hours, then the mixture was filtered and the cake washed with water (2928 ml) and dried at 60° C. to afford N₁-(2-oxopropyl)-N₂-(3-(trifluoromethyl)phenyl)oxalamide (370.0 g, purity=98.4%, yield=80.0%); ¹H NMR: (500 MHz CDCl₃): 2.3 (s, 3H), 4.25 (d, 2H), 7.44 (d, 1H), 7.50 (t, 1H), 7.83 (d, 1H), 7.98 (s, 1H), 8.18 (s, 1H), 9.31 (s, 1H).

### 4d: 6-Methyl-1-(3-(trifluoromethyl)phenyl)pyrazine-2,3(1H,4H)-dione

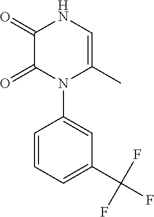

Concentrated sulphuric acid (98.0 g) was charged into a stirred flask and the mixture heated to 50-55° C. and at 50-55° C. N₁-(2-oxopropyl)-N₂-(3-(trifluoromethyl)phenyl)oxalamide (400.0 g) as 5 batches (80 g). The interval between two batches was about 2 to 5 minutes. After the addition, the reaction was held for 0.5 to 2 hours, then cooled to below 30° C. and transferred to another flask containing ice-water (8 kg ice and 4 kg water) at 0-5° C. The product was isolated by filtration and washed with cold water (1.2 kg×5) chilled to 0-5° C. Dried under 60° C. to yield 6-methyl-1-(3-(trifluoromethyl)phenyl)pyrazine-2,3(1H,4H)-dione (338.0 g, purity=98.5%, yield=90.3%); ¹H NMR: (500 MHz CDCl₃): 1.73 (s, 3H), 6.33 (s, 1H), 7.45 (d, 1H), 7.51 (s, 1H), 7.68 (t, 1H), 7.75 (d, 1H), 11.39 (s, 1H).

### 4e: 3-Bromo-6-methyl-1-(3-(trifluoromethyl)phenyl)pyrazin-2(1H)-one

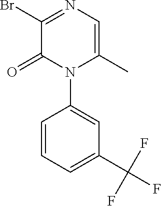

Acetonitrile (3300 ml) and 6-methyl-1-(3-(trifluoromethyl)phenyl)pyrazine-2,3(1H,4H)-dione (330.0 g) was charged to a flask and the mixture was warmed to 64-67° C. Phosphorus oxybromide (385.2 g) solution in acetonitrile (925.0 ml) was then added into the flask maintaining the temperature at 64-67° C., after the addition, keeping the reaction for 4 to 6 hours at 64-67° C. until the content of starting material<1%. Then the mixture was cooled to 0-10° C. and 4.42% aq. NaHCO₃ (12840 g) was charged for quenching, stirred the mixture for another 2 to 4 hours at 5-10° C. The mixture was filtered and the cake washed with water (990 ml), followed by drying at 65° C. to afford 3-bromo-6-methyl-1-(3-(trifluoromethyl)phenyl)pyrazin-2(1H)-one (342.5 g, purity=99.3%, yield=84.2%). The product was purified further as follows. 3-Bromo-6-methyl-1-(3-(trifluoromethyl)phenyl)pyrazin-2(1H)-one (30 kg) was dissolved in EtOAc (460 kg) in a 1000 L glass-lined reactor at 15-25° C., and then the mixture heated to 25-30° C. and stirred to make the mixture dissolve completely. Water (105 kg) was added and stirring continued for 30 min, held for 30 min and separated. The organic layer was washed with 13% brine (92 kg) and the organic phase was then concentrated under reduced pressure (temperature less than 55° C., pressure less than −0.08 MPa) until the residue was about 60 L. Petrol ether (200 kg) was added to the residue and cooled the mixture to 0-10° C. and stirred for 4 to 6 hours, followed by filtration and drying below 60° C. until LOD<0.5%. This gave a yellow powder (25.4 kg, purity=99.5%, yield=84.7%); ¹H NMR: (500 MHz CDCl₃): 1.93 (s, 3H), 7.14 (s, 1H), 7.43 (d, 1H), 7.51 (s, 1H), 7.72 (t, 1H), 7.80 (d, 1H).

### 4f; 4-(1H-pyrazol-1-yl)benzonitrile

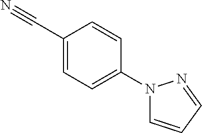

DMF (123.25 L was charged to the vessel and analysed for moisture content (target<0.5%). Potassium carbonate (34.01 kg) was then charged to the vessel followed by pyrazole (16.76 kg) and 4-fluorobenzonitrile (24.65 kg). The reaction mixture was heated to 115 to 120° C. and stirred at this temperature for 7 to 8 hours under a nitrogen atmosphere. The reaction was monitored by GC (target<10% 4-fluorobenzonitrile). The reaction was then cooled to 20-25° C. and quenched with water (369.7 L). MTBE (246.5 L) was then charged and the layers allowed to separate. The aqueous layer was washed with MTBE (2×147.9 L) and the organic layers combined. The combined organic layers were then washed with water (2×172.55 L) and aqueous brine (123.25 L, 24 wt %). The organic phase was then concentrated to approximately 100 L at 60° C. or below at atmospheric pressure. n-Heptane (209 L) was then charged and the mixture concentrated to approximately 100 L at 60° C. or below at atmospheric pressure. The reaction was cooled to 0° C. and stirred for 3 hours at this temperature. The slurry was then filtered washing the filter cake with n-heptane (24.65 L). The resulting solid was dried under vacuum at 40° C. to yield 4-(1H-pyrazol-1-yl)benzonitrile 28.6 kg, 99.32% purity, 83% yield.

### 4g: 4-[5-(4,4,5,5-Tetramethyl-[1,3,2]dioxaborolan-2-yl)-pyrazol-1-yl]-benzonitrile

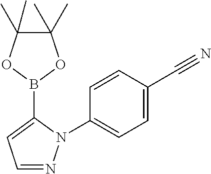

2,2,6,6-Tetramethylpiperidine (623.4 ml, 1.25 eq) and THF (2.5 L) were added to flask and cooled to −20+/−2° C. Hexyl lithium (2.3M, 1.542 L, 1.2 eq) was added over 2 hrs 20 min. maintaining internal temperature at −20+/−2° C. After complete addition, reaction was stirred at −20+/−2° C. for 30 minutes. The mixture was then cooled to −50+/−2° C., and a solution of 325 benzonitrile in THF (2.4 L) was then added slowly over 2 hours 23 minutes, keeping the temperature at −50+/−2° C. After addition was complete the mixture was stirred at −50+/−2° C. for 2.5 hours. Isopropyl pinacol borate (753.4 ml, 1.25 eq) was added to reaction mixture over 1 hour 6 minutes keeping the temperature at −50+/−2° C., followed by a line-wash of THF (0.3 L). After addition was complete the mixture was left to stir for 45 minutes, then allowed to warm to −15° C. Acetic acid (0.51 L, 1 eq) was added over 45 minutes keeping the temperature below 0° C. then stirred for 30 minutes at 0 to −5° C. Water (1.5 L) was then added over 1.5 hours keeping the temperature between 0 and −5° C. followed by the further water (4.5 L) over 1 hour. The mixture was then stirred between 0 and −5° C. for 30 minutes, and the product was filtered off, washed with cold water (1000 mL) four times and dried in a vacuum oven at 40° C. to constant weight to give 4-[5-(4,4,5,5-Tetramethyl-[1,3,2]dioxaborolan-2-yl)-pyrazol-1-yl]-benzonitrile, 566 g (64% Yield).

### 4h): 5-Methyl-3-oxo-4-(3-trifluoromethyl-phenyl)-3,4-dihydro-pyrazine-2-carboxylic acid methyl ester

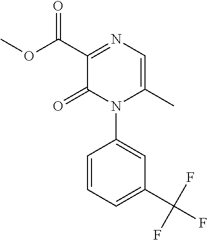

3-Bromo-6-methyl-1-(3-trifluoromethyl-phenyl)-1H-pyrazin-2-one (750 g), diacetoxypalladium (3 g, 0.006 eq), 1,3-bis(diphenylphosphino)propane (6.6 g, 0.007 eq) and triethylamine (600 ml) were dissolved in methanol (3.15 L). The reaction mixture was degassed with carbon monoxide (10 bar) and heated to 65° C. for 12 hours. The reaction mixture was concentrated to ⅔ of its original volume and cooled to 0° C. The product was filtered, washed three times with methanol and diethyl ether (1 L). 5-Methyl-3-oxo-4-(3-trifluoromethyl-phenyl)-3,4-dihydro-pyrazine-2-carboxylic acid methyl ester was slurried in water (2 L), filtered and was dried in under vacuum to give 590 g (85% Yield).

### 4i: 6-Bromo-5-methyl-3-oxo-4-(3-trifluoromethyl-phenyl)-3,4-dihydro-pyrazine-2-carboxylic acid methyl ester

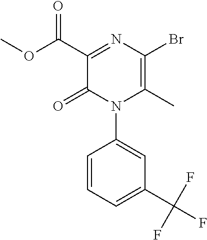

5-Methyl-3-oxo-4-(3-trifluoromethyl-phenyl)-3,4-dihydro-pyrazine-2-carboxylic acid methyl ester (400 g, 1 eq.) was dissolved in dimethylformamide (3 L) and stirred at 17-20° C. (N-Bromosuccinimide (229.3 g, 1 eq) was dissolved in dimethyl formamide (1 L), and added to the ester solution over 1 hour. Post-addition, the reaction was stirred at 17-20° C. for 10 hours. The reaction mixture was added to water (15 L) with stirring. The resulting slurry was stirred at 20-25° C. overnight. 6-Bromo-5-methyl-3-oxo-4-(3-trifluoromethyl-phenyl)-3,4-dihydro-pyrazine-2-carboxylic acid methyl ester was collected by filtration. The cake was washed with water (1 L) and heptane (1 L). Dried to constant weight at 40° C. to give 456 g (91% Yield).

### 4j: 6-[2-(4-Cyano-phenyl)-2H-pyrazol-3-yl]-5-methyl-3-oxo-4-(3-trifluoromethyl-phenyl)-3,4-dihydro-pyrazine-2-carboxylic acid methyl ester

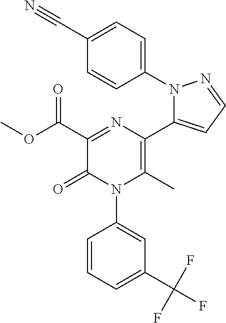

6-Bromo-5-methyl-3-oxo-4-(3-trifluoromethyl-phenyl)-3,4-dihydro-pyrazine-2-carboxylic acid methyl ester (435 g, 1.11 mol), 4-[5-(4,4,5,5-tetramethyl-[1,3,2]dioxaborolan-2-yl)-pyrazol-1-yl]-benzonitrile (503 g, 1.536 eq), sodium acetate (273.1 g, 3 eq), dichloro 1,1-bis(di-tert-butylphosphino)ferrocene palladium (36.1 g, 0.05 eq) and dimethylformamide (4.35 L) were charged to reaction vessel under an inert atmosphere, and heated to 50° C. Once at temperature, water (20 ml, 1 eq) was added, and mixture stirred for 9 hours. Reaction mixture was allowed to cool to 20-25° C., and was added to water (21.8 L) over a 2 hour period. The mixture was stirred at 20-25° C. for 30 minutes, and the product was isolated by filtration. The cake was washed with water (2×4.3 L) and tert-butyl methyl ether (2×4.3 L) and dried overnight under vacuum at 20-25° C. to give 493 g of crude 6-[2-(4-Cyano-phenyl)-2H-pyrazol-3-yl]-5-methyl-3-oxo-4-(3-trifluoromethyl-phenyl)-3,4-dihydro-pyrazine-2-carboxylic acid methyl ester.

The crude product (493 g) was further purified by dissolution in acetonitrile (9.7 L) and passage through two CUNO filters. The filters were washed with acetonitrile (2×5 L).

The combined organic phases were treated with Smopex® 111 scavenger (98.6 g), stirring at 50° C. for 10 hours before filtering through silica (60 Å, 230-400 mesh, 2.46 Kg). The silica was washed again with acetonitrile (2×4.9 L).

The acetonitrile solution was concentrated to about 2.5 L. Tert-butyl methyl ether (5 L) was added, and removed by distillation. This was repeated twice more. The resulting slurry was filtered, and the product washed with tert-butyl methyl ether (1 L) to give the title product 398.5 g (73% Yield).

### 4k: 6-[2-(4-Cyano-phenyl)-2H-pyrazol-3-yl]-5-methyl-3-oxo-4-(3-trifluoromethyl-phenyl)-3,4-dihydro-pyrazine-2-carboxylic acid ethylamide Form B (Compound (I) Form B)

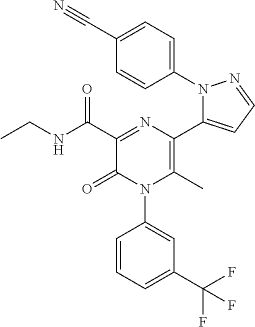

To 6-[2-(4-Cyano-phenyl)-2H-pyrazol-3-yl]-5-methyl-3-oxo-4-(3-trifluoromethyl-phenyl)-3,4-dihydro-pyrazine-2-carboxylic acid methyl ester (3.50 kg) was added ethylamine (2.0 M in MeOH) (28.6 kg, 36.5 L, 10.4 vol., 10.0 eq.) in one portion followed by a methanol line wash (3.5 kg, 4.4 L). The yellow suspension was stirred at 20±5° C. for 18 to 22 hours (colour changes from yellow to green to brown/purple). The product mixture was cooled to 5±3° C., filtered and top washed with cold methanol (at 6±4° C.) (2×27.7 kg, 2×35 L, 2×10 vol). The product was deliquored and then dried further under vacuum at 35 to 45° C. The dried solid (2.9 kg) was then dissolved in MIBK (69.7 kg, 30 vol. with respect to dried solid) at 60 to 70° C. (if solution did not form after 2 hours, the mixture was heated to 70 to 80° C. for a further 1-2 hours) and then polish-filtered maintaining a temperature>30° C. A line rinse through the filter was carried out using MIBK (approximately 8 kg, 10 L). The solution was cooled to 30 to 40° C. and the MIBK was distilled off under vacuum until about 6 vol. remained (at a temperature of <40° C.). The product was slurried in MIBK (17.4 L, about 6 vol.) for 14 to 18 hours at 6±4° C. and then filtered, deliquored and top-washed with cold MIBK (11.6 kg, 14.5 L, 5 vol.). The final product was further dried at 45 to 55° C. under vacuum to give Compound (I) Form B (2.26 kg, 63% yield).

The Compound (I) Form B may be further purified as follows. To solid Compound (I) Form B (1.76 kg) was added MIBK (8.65 kg, 10.8 L, 6 vol.) (through a 0.6 micron filter) and water (suitable for injection) (5% w/v. with respect to MIBK, 0.53 kg, 0.53 L) and the mixture was heated at 60±5° C. for at least 24 hours (24-80 hours). The slurry was then cooled to 5±5° C. over a period of at least 1 hour and then cooled to 5±5° C. with a hold at this temperature for at least 30 minutes. The slurry was filtered and deliquored. It was then top-washed with cold (5±5° C.) MIBK (7.21 kg, 9.0 L, 5 vol.) and deliquored again. The final product was further dried at 45 to 55° C. under vacuum until at constant weight to yield 6-[2-(4-cyano-phenyl)-2H-pyrazol-3-yl]-5-methyl-3-oxo-4-(3-trifluoromethyl-phenyl)-3,4-dihydro-pyrazine-2-carboxylic acid ethylamide Form B; 81% yield, >99% purity; ¹H NMR: (400 MHz d6-DMSO): 1.03 (t, 3H); 1.88 (s, 3H); 3.20 (m, 2H); 6.97 (d, 1H); 7.68 (m, 2H); 7.79 (d, 1H); 7.92 (m, 6H); 8.73 (t, 1H) (measured using Bruker 400 MHz NMR, using the residual solvent peaks as internal reference).

### Example 5

### Preparation of Compound (I) Form B

### 5a: Ethyl 2-oxo-2-(3-(trifluoromethyl)phenylamino)acetate

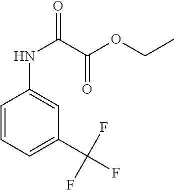

3-(Trifluoromethyl) aniline (56.35 g), triethylamine (49.5 g) and ethyl acetate (507.2 mL) were charged into the flask and cooled to 0 to 5° C., then ethyl oxalyl chloride (57.33 g) was added dropwise, keeping the reaction temperature at 0 to 10° C. When the addition was complete, the mixture was stirred for thirty minutes at 0 to 10° C. The mixture was warmed to 15 to 25° C. and held for between one and two hours then sampled every two hours until the content of starting material was <1%. Water (282 mL) was added dropwise slowly at 15 to 25° C. to quench the reaction. After the addition, the mixture was stirred for another thirty minutes and held for thirty minutes before separation. The mixture was separated and the aqueous phase extracted with ethyl acetate (125.1 ml). The organic phases were combined and washed with saturated brine (115 ml) after water (140.9 ml). The organic phase was concentrated under reduced pressure (<50° C., pressure<−0.08 MPa), n-heptane (56.35 g) added and the mixture concentrated further until the wt % of ethyl acetate was <20%. Then petrol ether (169.1 ml) was added to the residue. The solids were filtered and the cake was washed with petrol ether (15.21 ml) chilled to 0 to 10° C. and dried at <50° C. to afford the title product (88.89 g, purity=98.9%, yield=97.3%); ¹H NMR: (500 MHz CDCl₃): 1.44 (t, 3H), 4.44 (q, 2H), 7.46 (d, 1H), 7.52 (t, 1H), 7.88 (d, 1H), 7.92 (s, 1H), 9.00 (s, 1H).

### 5b: N₁-(2-hydroxypropyl)-N₂-(3-(trifluoromethyl)phenyl)oxalamide

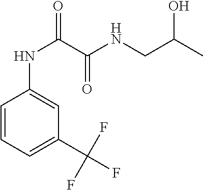

Ethyl 2-oxo-2-(3-(trifluoromethyl)phenylamino)acetate (50.0 g) and anhydrous ethanol (250 ml) were charged into a flask and the mixture was heated to 70-75° C. at the rate of 40-45° C./hour to give a gentle reflux, then 1-amino-2-propanol (15.8 g) was added at the rate of 0.9 g-1.0 g/minute, while maintaining the temperature at 70-75° C. After the addition, the mixture was held for between two and three hours at 70-75° C. The mixture was then cooled to <50° C. and concentrated until the wt % of ethanol was <20%. n-Heptane (265 ml) was added. The mixture was cooled to 0-5° C. and stirred for between two and three hours. The solids were filtered and the cake was washed with n-heptane (16 ml), then dried under 50° C. to afford the title product (47.2 g, purity=97.5%, yield=85.0%); ¹H NMR: (500 MHz CDCl₃): 1.26 (d, 3H), 2.30 (s, 1H), 3.28 (m, 1H), 3.56 (m, 1H), 4.03 (m, 1H), 7.43 (d, 1H), 7.47 (t, 1H), 7.81 (d, 1H), 7.99 (s, 1H), 8.01 (s, 1H), 9.52 (s, 1H).

### 5c: N₁-(2-oxopropyl)-N₂-(3-(trifluoromethyl)phenyl)oxalamide

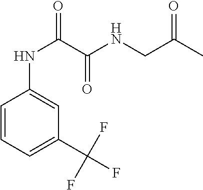

N₁-(2-hydroxypropyl)-N₂-(3-(trifluoromethyl)phenyl)oxalamide (20.0 g), acetonitrile (200 ml) and ruthenium chloride hydrate (0.208 g) in water (6.0 ml) were charged to the flask and the contents maintained at 10-30° C. A solution of sodium bromate (11.46 g) in water (48.0 ml) was added into the mixture. After the addition, the mixture was held at 20-25° C. for four hours. Water (300 ml) was added to the mixture and stirred at 0-10° C. for between two and three hours. The mixture was filtered and the cake washed with water (300 ml) and dried at 60° C. to afford the title product (18.1 g, purity=95.5%, yield=91.1%); ¹H NMR: (500 MHz CDCl₃+50 μL DMSO-d6): 2.23 (s, 3H), 4.20 (d, 2H), 7.40 (d, 1H), 7.49 (t, 1H), 7.95 (d, 1H), 8.21 (s, 1H), 8.68 (t, 1H), 10.32 (s, 1H).

### 5d: 6-Methyl-1-(3-(trifluoromethyl)phenyl)pyrazine-2,3(1H,4H)-dione

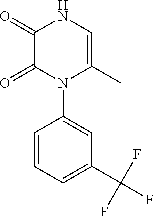

Concentrated sulphuric acid (300 ml) was charged into a flask and heated to 50-55° C. N₁-(2-oxopropyl)-N₂-(3-(trifluoromethyl)phenyl)oxalamide (100.0 g) was charged in three portions, maintaining the temperature at 50-55° C. After the addition, the reaction was held at 50-55° C. for between two and three hours, then cooled to below 30° C. and transferred to another flask containing ice-water (900 g ice and 450 ml water) at 0-5° C. The product was isolated by filtration and washed with cold water (2×250 g) chilled to 0-5° C. The filter cake was then washed with anhydrous ethanol (100.0 g) and dried under 60° C. to yield the title product (84.4 g, purity=99.4%, yield=90.0%); ¹H NMR: (500 MHz CDCl₃): 1.72 (s, 3H), 6.39 (s, 1H), 7.45 (d, 1H), 7.51 (s, 1H), 7.68 (t, 1H), 7.75 (d, 1H), 11.63 (s, 1H).

### 5e: 3-Bromo-6-methyl-1-(3-(trifluoromethyl)phenyl)pyrazin-2(1H)-one

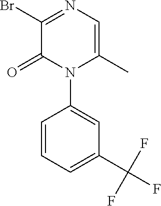

Acetonitrile (70 ml) and 6-methyl-1-(3-(trifluoromethyl)phenyl)pyrazine-2,3(1H,4H)-dione (10.0 g) were charged to a flask and the mixture was warmed to 50-55° C. The water content of the solution was checked by KF to be ≦0.3%. A solution of phosphorus oxybromide (13.26 g) in acetonitrile (53 ml) was then added into the flask maintaining the temperature at 64-67° C. The reaction was held for between four and six hours at 64-67° C. until the content of starting material was <1%. The mixture was cooled to 0-10° C. and a solution of NaHCO₃ (19.43 g) in water (370 ml) was charged for quenching. The mixture was stirred for between two and four hours at 0-10° C. The mixture was filtered and the cake washed with water (30 ml) to afford the crude title product (10.6 g, purity=99.2%, yield=86.0%). The crude product was purified further as follows.

3-Bromo-6-methyl-1-(3-(trifluoromethyl)phenyl)pyrazin-2(1H)-one (30 kg) was dissolved in EtOAc (460 kg) in a 1000 L glass-lined reactor at 15-25° C. The mixture was heated to 25-30° C. and stirred to make the mixture dissolve completely. Water (105 kg) was added and stirring continued for thirty minutes. The biphasic mixture was held for thirty minutes and separated. The organic layer was washed with 13% brine (92 kg) and the organic phase was then concentrated under reduced pressure (temperature less than 55° C., pressure less than −0.08 MPa) until the residue was about 60 L. Petrol ether (200 kg) was added to the residue and cooled the mixture to 0-10° C. and stirred for between four and six hours, followed by filtration and drying below 60° C. until LOD<0.5%. This gave the title product as a yellow powder (25.4 kg, purity=99.5%, yield=84.7%); ¹H NMR: (500 MHz CDCl₃): 1.93 (s, 3H), 7.14 (s, 1H), 7.43 (d, 1H), 7.51 (s, 1H), 7.72 (t, 1H), 7.80 (d, 1H).

### 5f: 4-(1H-pyrazol-1-yl)benzonitrile

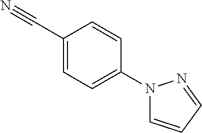

N,N-dimethylformamide (123.25 L) was charged to the vessel and analysed for moisture content (target<0.5%). Potassium carbonate (34.01 kg) was then charged to the vessel followed by pyrazole (16.76 kg) and 4-fluorobenzonitrile (24.65 kg). The reaction mixture was heated to 115 to 120° C. and stirred at this temperature for between seven and eight hours under a nitrogen atmosphere. The reaction was monitored by GC (target<10% 4-fluorobenzonitrile). The reaction was then cooled to 20-25° C. and quenched with water (369.7 L). Methyl tert-butyl ether (246.5 L) was then charged and the layers allowed to separate. The aqueous layer was washed with methyl tert-butyl ether (2×147.9 L) and the organic layers combined. The organic layer was then washed with water (2×172.55 L) and aqueous brine (123.25 L, 24 wt %). The organic phase was then concentrated to approximately 100 L at 60° C. or below at atmospheric pressure. n-Heptane (209 L) was then charged and the mixture concentrated to approximately 100 L at 60° C. or below at atmospheric pressure. The reaction was cooled to 0° C. and stirred for three hours at this temperature. The slurry was then filtered washing the filter cake with n-heptane (24.65 L). The resulting solid was dried under vacuum at 40° C. to yield the title product (28.6 kg, 99.32% purity, 83% yield). ¹H NMR (300 MHz, DMSO-d6): 6.61 (dd, J=2.4, 1.9 Hz, 1H), 7.83 (d, J=1.4 Hz 1H), 7.94 (d, J=8.7 Hz, 2H), 8.04 (d, J=9.0 Hz, 2H), 8.65 (d, J=2.4 Hz, 1H).

### 5g: 4-[5-(4,4,5,5-Tetramethyl-[1,3,2]dioxaborolan-2-yl)-pyrazol-1-yl]-benzonitrile

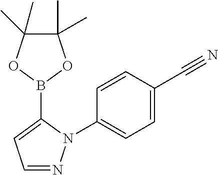

To 2,2,6,6-tetramethylpiperidine (24.07 g) was added tetrahydrofuran (115 mL). The reaction mixture was cooled to −20° C. At this temperature 1.6 M n-butyl lithium in hexanes (102 mL) was added, maintaining the temperature at less than 0° C. The reaction mixture was stirred at 0° C. for thirty minutes. The reaction mixture was cooled to −50° C. At this temperature a pre-mixed solution of 4-(1H-pyrazol-1-yl)benzonitrile (23 g) dissolved in tetrahydrofuran (115 mL) was added over approximately ten minutes, maintaining the temperature at less than −50° C. Tetrahydrofuran (12 mL) was added, and the reaction was stirred at −50° C. for at least thirty minutes. 2-isopropoxy-4,4,5,5-tetramethyl-1,3,2-dioxaborolane (35 mL) was charged over approximately twenty-five minutes, whilst maintaining the temperature at less than −50° C. The reaction mixture was stirred for approximately thirty minutes at −50° C., and then allowed to warm to approximately −15° C. Acetic acid (23.5 mL) was added over approximately twenty minutes, maintaining the temperature at less than 0° C. For convenience the reaction mixture was allowed to warm to approximately 20° C. over approximately sixteen hours. The reaction mixture was cooled to 0° C. and water (276 mL) was charged over approximately thirty-five minutes, maintaining the temperature at less than 0° C. The reaction mixture was stirred for an additional thirty minutes. The product was isolated by filtration and washed (on the filter) with water (46 mL) and then with n-heptane (46 mL). The damp solid was then dried in vacuo at 40° C. for approximately twenty-four hours to give the title product (34.62 g, 86.28%); ¹H NMR (500 MHz, CDCl₃% 1.23 (s, 12H), 6.90 (d, J=1.7 Hz, 1H), 7.69-7.63 (m, 4H), 7.70 (d, J=1.7 Hz, 1H). ¹³C NMR (125 MHz, CDCl₃): 143.4, 140.4, 131.5, 123.8, 118.4, 117.5, 109.7, 83.7, 67.0, 23.6.

### 5h): 5-Methyl-3-oxo-4-(3-trifluoromethyl-phenyl)-3,4-dihydro-pyrazine-2-carboxylic acid methyl ester

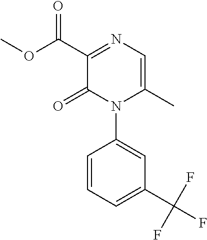

3-Bromo-6-methyl-1-(3-trifluoromethyl-phenyl)-1H-pyrazin-2-one (1332.0 g), diacetoxypalladium (6.0 g), 1,3-bis(diphenylphosphino)propane (12.0 g), triethylamine (809.6 g) and methanol (5.1 kg) were charged into the reactor. The mixture was exchanged with nitrogen gas eight times and exchanged with carbon monoxide five times, then ventilated with carbon monoxide to a pressure of 1.5 MPa. The mixture was heated to 60-65° C. and stirred at 60-65° C. for ten hours. The mixture was cooled to 20-30° C., and the mixture was exchanged with nitrogen eight times. Then it was concentrated at ≦45° C. under reduced pressure (P≦−0.08 MPa). The residual mixture was cooled to 0-5° C. and stirred at this temperature for thirty minutes and filtered. The filter cake was washed with pre-mixed methanol (288.4 g) and methyl tert-butyl ether (270.1 g) and then was washed with water (3.65 kg). The filter cake was washed with the mixed solvent of methanol (434.5 g) and methyl tert-butyl ether (407.0 g) again, before drying at ≦50° C. to afford the title product (1073.0 g, purity=99.2%, yield=86.0%); ¹H NMR: (500 MHz CDCl₃): 2.06 (s, 3H), 3.97 (s, 3H), 7.43 (d, 1H), 7.47 (s, 1H), 7.51 (s, 1H), 7.73 (t, 1H), 7.81 (d, 1H).

### 5i: 6-Bromo-5-methyl-3-oxo-4-(3-trifluoromethyl-phenyl)-3,4-dihydro-pyrazine-2-carboxylic acid methyl ester

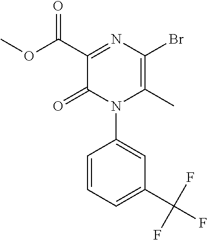

5-Methyl-3-oxo-4-(3-trifluoromethyl-phenyl)-3,4-dihydro-pyrazine-2-carboxylic acid methyl ester (998.4 g) and N,N-dimethylformamide (6.4 kg) were charged into the flask and the mixture was heated to 35-40° C. until the solid dissolved completely, then the mixture was cooled to 17-23° C. A solution of N-bromosuccinimide (626.6 g) in N,N-dimethylformamide (2.35 kg) was added dropwise at 17-23° C. The mixture was stirred at 17-23° C. for three hours. The mixture was poured into water (40.0 kg) and stirred at 17-23° C. for between one and two hours. The product was collected by filtration, and the filter cake was washed three times with water (3×1333.0 g) and then washed twice with n-Heptane (2×1.36 kg). The filter cake was dried under 50° C. to afford the crude title product (1158.7 g, purity=96.0%, yield=92.6%); ¹H NMR: (500 MHz CDCl₃): 2.23 (s, 3H), 3.95 (s, 3H), 7.40 (d, 1H), 7.49 (s, 1H), 7.73 (t, 1H), 7.81 (d, 1H).

The crude product was purified further as follows.

6-Bromo-5-methyl-3-oxo-4-(3-trifluoromethyl-phenyl)-3,4-dihydro-pyrazine-2-carboxylic acid methyl ester (121.4 kg) was dissolved in methanol (238.0 kg) and the mixture was heated to 58-63° C. and stirred at this temperature for between thirty minutes and one hour. The mixture was then cooled to 0-5° C. and stirred at this temperature for between two and four hours. The product was collected by filtration to afford the title product as a yellow solid (102.0 kg, purity=99.3%, yield=84.0%).

### 5j: 6-[2-(4-Cyano-phenyl)-2H-pyrazol-3-yl]-5-methyl-3-oxo-4-(3-trifluoromethyl-phenyl)-3,4-dihydro-pyrazine-2-carboxylic acid methyl ester

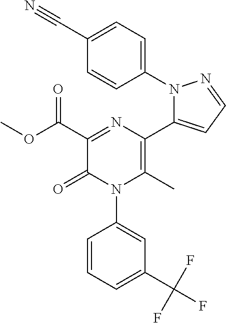

To a nitrogen-purged vessel was added 6-bromo-5-methyl-3-oxo-4-(3-trifluoromethyl-phenyl)-3,4-dihydro-pyrazine-2-carboxylic acid methyl ester (12.50 g), 4-[5-(4,4,5,5-tetramethyl-[1,3,2]dioxaborolan-2-yl)-pyrazol-1-yl]-benzonitrile (10.38 g), degassed 4-methylpentan-2-one (250 mL), triethylamine (13.36 mL), 1,1′ bis(di-tert-butylphosphino)ferrocene palladium(II) dichloride (210.0 mg) and water (1.15 mL). The reaction mixture was heated to 80° C. over one hour, and maintained at this temperature for ten hours before cooling to 20° C. The reaction mixture was reduced to approximately half the volume by reduced pressure distillation (at less than 70° C.). The reaction temperature was adjusted to 5° C., then left stirring for a further two hours. The product was isolated by filtration and washed (on the filter) with methanol (50 mL). The damp solid was then dried in vacuo overnight at 40° C. This provided the sub-titled compound as a yellow solid (12.70 g; 81.2% yield); ¹H-NMR (500 MHz, DMSO-d6): 1.92 (s, 3H), 3.71 (s, 3H), 6.77 (d, J=1.3 Hz, 1H), 7.68 (d, J=8.5 Hz, 2H), 7.82 (d, J=7.9 Hz, 1H), 7.86-7.94 (m, 4H), 7.96 (d, J=7.9 Hz, 1H), 7.99 (s, 1H).

### 5k: 6-[2-(4-Cyano-phenyl)-2H-pyrazol-3-yl]-5-methyl-3-oxo-4-(3-trifluoromethyl-phenyl)-3,4-dihydro-pyrazine-2-carboxylic acid ethylamide Form A (Compound (I) Form A)

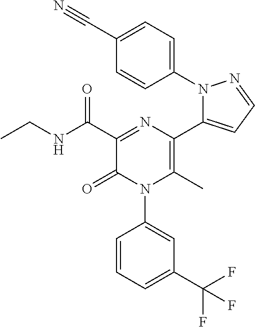

Methanol (290 mL) and acetonitrile (38 mL) were charged to the reactor and chilled to 5° C. Ethylamine, 70% in water (89 mL) was added over approximately thirty minutes, maintaining the temperature below 10° C. 6-[2-(4-Cyano-phenyl)-2H-pyrazol-3-yl]-5-methyl-3-oxo-4-(3-trifluoromethyl-phenyl)-3,4-dihydro-pyrazine-2-carboxylic acid methyl ester (46.14 g) was then charged in one portion, followed by methanol (46 mL). The reaction mixture was stirred at 5° C. for approximately twenty-four hours. The product was isolated by filtration and washed twice with chilled, pre-mixed methanol (115 mL) and water (115 mL). The damp solid was dried in vacuo to constant weight at 40° C. to provide the titled product as a yellow solid (41.43 g, 95.70% purity, 85.36% yield); ¹H-NMR (500 MHz, DMSO-d6): ¹H NMR (500 MHz, DMSO-d6) 1.02 (t, J=7.2 Hz, 3H), 1.88 (s, 3H), 3.14-3.25 (m, 2H), 6.77 (d, J=1.4 Hz, 1H), 7.68 (d, J=8.6 Hz, 2H), 7.78 (d, J=7.9 Hz, 1H), 7.85-7.92 (m, 3H), 7.92-7.99 (m, 3H), 8.71 (t, J=5.5 Hz, 1H).

### 5l: 6-[2-(4-Cyano-phenyl)-2H-pyrazol-3-yl]-5-methyl-3-oxo-4-(3-trifluoromethyl-phenyl)-3,4-dihydro-pyrazine-2-carboxylic acid ethylamide Form B (Compound (I) Form B)

To 6-[2-(4-Cyano-phenyl)-2H-pyrazol-3-yl]-5-methyl-3-oxo-4-(3-trifluoromethyl-phenyl)-3,4-dihydro-pyrazine-2-carboxylic acid ethylamide Form A (18.34 g) was added methyl isobutyl ketone (220 mL). The resulting mixture was heated to 85° C. over sixty-five minutes and held at this temperature for approximately forty-five minutes. The resulting solution was filtered hot and washed through with methyl isobutyl ketone (19 mL). The temperature was adjusted to 60° C. and solvent (114 mL collected in receiver) was removed by distillation under reduced pressure. The resulting product slurry was cooled to −5° C. over approximately five and a half hours, and then left stirring overnight. The product was isolated by filtration and washed twice with chilled methyl isobutyl ketone (46 mL). The damp solid was dried in vacuo to constant weight at 45° C. to provide the title product as a yellow solid (15.20 g, 99.30% purity, 82.88% yield).

The 6-[2-(4-Cyano-phenyl)-2H-pyrazol-3-yl]-5-methyl-3-oxo-4-(3-trifluoromethyl-phenyl)-3,4-dihydro-pyrazine-2-carboxylic acid ethylamide Form B (Compound (I) Form B) may be further purified as follows.

To solid Compound (I) Form B (1.76 kg) was added MIBK (8.65 kg, 10.8 L, 6 vol.) (through a 0.6 micron filter) and water (suitable for injection) (5% w/v. with respect to MIBK, 0.53 kg, 0.53 L) and the mixture was heated at 60±5° C. for at least 24 hours (24-80 hours). The slurry was then cooled to 5±5° C. over a period of at least 1 hour and then cooled to 5±5° C. with a hold at this temperature for at least 30 minutes. The slurry was filtered and deliquored. It was then top-washed with cold (5±5° C.) MIBK (7.21 kg, 9.0 L, 5 vol.) and deliquored again. The final product was further dried at 45 to 55° C. under vacuum until at constant weight to yield Compound (I) Form B; 81% yield, >99% purity; ¹H NMR: (400 MHz d6-DMSO): 1.03 (t, 3H); 1.88 (s, 3H); 3.20 (m, 2H); 6.97 (d, 1H); 7.68 (m, 2H); 7.79 (d, 1H); 7.92 (m, 6H); 8.73 (t, 1H) (measured using Bruker 400 MHz NMR, using the residual solvent peaks as internal reference).

### Summary of Characteristics of Compound (I) Form B.

X-ray powder diffraction analysis of Compound (I) Form B gave the XRPD pattern substantially as shown in FIG. 3 measured under ambient conditions (nickel-filtered CuK-radiation (1.5418 Å, 45 kV, 40 mA)).

The hygroscopicity of Compound (I) Form B, defined as the water uptake by a dried sample when the relative humidity is increased from 0% to 80% at 25° C., was 0.08% (W/W).

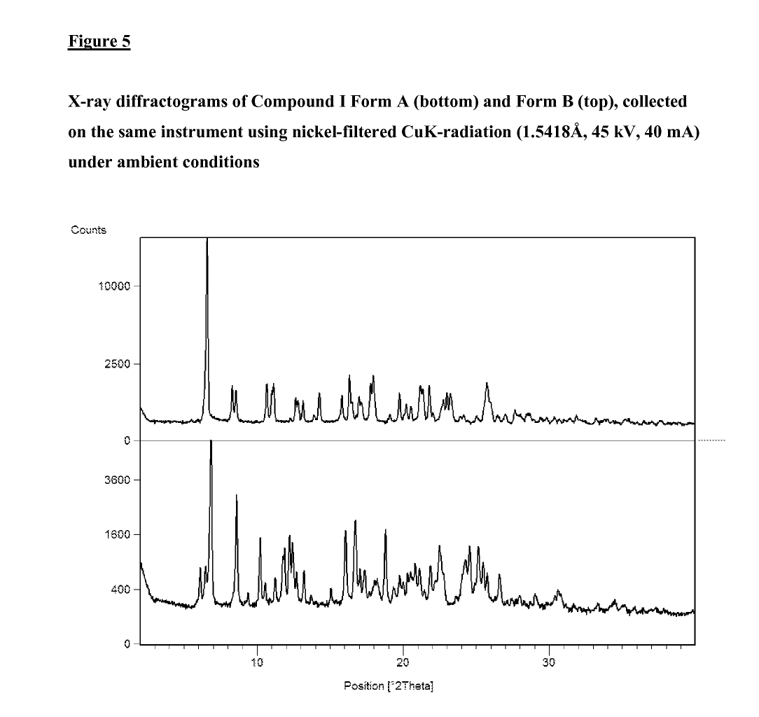

X-ray powder diffraction analysis of compound (I) Form A and Form B using the same instrument (PANalytical X'Pert PRO MPD theta-theta system using nickel-filtered CuK-radiation (1.5418 Å, 45 kV, 40 mA) and an X'Celerator detector), under ambient conditions gave the XRPD pattern substantially as shown in FIG. 5. It is thought that the unit cell of Form A varies slightly depending on the relative humidity it is exposed to. Therefore the XRPD pattern for Form A in FIG. 6 is slightly different to the XRPD pattern for Form A shown in FIG. 1, measured under controlled temperature and humidity conditions.

### Example 6

### Dry Powder Composition

Compound (I) Form B (4 kg) was micronized in a 4″ jet mill at a feed rate of 4 kg/h with a milling pressure of 4.5 bar and ejector pressure of 6 bar. The resulting particles had a mass median diameter (MMD) of 1.8-2.1 μm (measured using a Malvern Master Sizer using miglyol as solvent). The micronized Compound (I) Form B was conditioned in ethanol vapor (activity 0.7 (100% ethanol at 19.0° C., increased in temperature to 25.0° C.)) for 24 hours. The resulting powder was added together with lactose monohydrate that had been micronised and conditioned using the method disclosed in WO 95/05805 (particle size measured with Coulter counter 2.4 μm) in a mixing drum (batch size 1.4 kg, drum size 17 L, 15 min, 24 rpm). The resulting mixture was passed through a spiral jet mill operating at a low chamber pressure (0.5 bar, feed rate 5 kg/h). The mixture (portions of 450-550 g) was then continuously added by a screw feeder to an oscillating sieve (mesh size 0.5 mm), followed by a process to form spherical aggregates by tumbling the material at 23 rpm for 4 min. The resulting aggregates were then passed through an intermediate sieve and collected for further strengthening by tumbling at 23 rpm for 6 min. A final sieving step with a mesh size of 0.8 mm gave the fraction of granules with a size less than 0.8 mm. The resulting spheronized granules may then be added to a suitable dry powder inhaler such as a Turbuhaler.

### Biological Activity

### Human Neutrophil Elastase Quenched-Fret Assay

The assay uses Human Neutrophil Elastase (HNE) purified from serum (Calbiochem art. 324681; Ref. Baugh, R. J. et al., 1976, Biochemistry. 15, 836-841). HNE was stored in 50 mM sodium acetate (NaOAc), 500 mM sodium chloride (NaCl), pH 5.5 with added 50% glycerol at −20° C. The protease substrate used was Elastase Substrate V Fluorogenic, MeOSuc-AAPV-AMC (Calbiochem art. 324740; Ref. Castillo, M. J. et al., 1979, Anal. Biochem. 99, 53-64). The substrate was stored in dimethyl sulfoxide (DMSO) at −20° C. The assay additions were as follows: Test compounds and controls were added to black 96-well flat-bottom plates (Greiner 655076), 1.0 μL in 100% DMSO, followed by 30 μL HNE in assay buffer with 0.01% Triton (trade mark) X-100 detergent. The assay buffer constitution was: 100 mM Tris(hydroxymethyl)aminomethane (TRIS) (pH 7.5) and 500 mM NaCl. The enzyme and the compounds were incubated at room temperature for 15 minutes. Then 30 μl substrate in assay buffer was added. The assay was incubated for 90 minutes at room temperature. The concentrations of HNE enzyme and substrate during the incubation were 0.17 nM and 100 μM, respectively. The assay was then stopped by adding 60 μl stop solution (140 mM acetic acid, 200 mM sodium monochloroacetate, 60 mM sodium acetate, pH 4.3). Fluorescence was measured on a Wallac EnVision instrument at settings: Excitation 380 nm, Emission 460 nm. IC₅₀ values were determined using Xlfit curve fitting using model 203.

When tested in the above assay, Compound (I) (dissolved in DMSO) gave an IC₅₀ value for inhibition of human neutrophil elastase activity of 0.27 nM (n=7).

1. The compound 6-[2-(4-Cyano-phenyl)-2H-pyrazol-3-yl]-5-methyl-3-oxo-4-(3-trifluoromethyl-phenyl)-3,4-dihydro-pyrazine-2-carboxylic acid ethylamide Form B.

2. The compound according to claim 1 wherein said Form B has an X-ray powder diffraction pattern with at least one specific peak at 2θ about =14.3 or 23.2° measured using CuK-radiation at 1.5418 Å.

3. The compound according to claim 1 wherein said Form B has an X-ray powder diffraction pattern with at least one specific peak at 2θ about =14.3, 17.8 or 23.2° measured using CuK-radiation at 1.5418 Å.

4. The compound according to claim 1 wherein said Form B has an X-ray powder diffraction pattern with specific peaks at 2θ about =6.6, 14.3, 17.8 and 23.2° measured using CuK-radiation at 1.5418 Å.

5. The compound according to claim 1 wherein said Form B has an X-ray powder diffraction pattern substantially as shown in FIG. 3, measured using CuK-radiation at 1.5418 Å.

8. A pharmaceutical composition comprising a compound according to claim 1 in admixture with a pharmaceutically acceptable diluent or carrier.

9. The pharmaceutical composition according to claim 8, which is formulated for inhaled administration.

10. The pharmaceutical composition according to claim 8, which is a dry powder composition comprising a pharmaceutically acceptable carrier and finely divided particles of the compound.

11. An inhaler containing a compound according to claim 1.

12. An inhaler containing a pharmaceutical composition according to claim 9.
